(19) United States
(12) Patent Application Publication
(10) Pub. No.: US 2025/0271444 A1
(43) Pub. Date: Aug. 28, 2025

(54) METHODS OF PROTEIN ANALYSIS

(71) Applicant: Calico Life Sciences LLC, South San Francisco, CA (US)

(72) Inventors: Fiona McAllister, South San Francisco, CA (US); Aleksandr Gaun, South San Francisco, CA (US); Niclas Olsson, South San Francisco, CA (US)

(21) Appl. No.: 18/256,907

(22) PCT Filed: Dec. 13, 2021
(86) PCT No.: PCT/US2021/063154

§ 371 Date: Jun. 9, 2023

Related U.S. Application Data
(60) Provisional application No. 63/124,651, filed on Dec. 11, 2020.

(51) Int. Cl. G01N 33/68

(52) U.S. Cl. CPC G01N 33/6848; G01N 2458/15

(57) ABSTRACT

Provided herein are methods of quantifying or identifying two or more peptides or polypeptides, methods of quantifying or identifying two or more chemically isotopic labeled peptides or polypeptides, and methods for obtaining a plurality of enriched peptides or polypeptides in a sample.

### CROSS REFERENCE TO RELATED APPLICATIONS

This application is the National Stage Entry of International Application No. PCT/US2021/063154 filed on Dec. 13, 2021, which claims the benefit of and priority to U.S. Provisional Patent Application No. 63/124,651 filed Dec. 11, 2020, the entire disclosures of each of which are hereby incorporated by reference in their entireties for all purposes.

### BACKGROUND

The proteome is usually described as the entire complement of proteins found in a biological system, such as, e.g., a cell, tissue, organ or organism. Proteomics is the study of the proteome expressed at particular times and/or under internal or external conditions of interest. Proteomics approaches frequently aim at global analysis of the proteome, and require that large numbers of proteins, e.g., hundreds or thousands, can be routinely resolved, identified and quantified from a single sample.

Analyses of proteins from large cohort samples are typically restricted to high-throughput automated assays using antibody-based or aptamer-based technologies. These assays are restricted to panel targets and are therefore not unbiased. Untargeted mass spectrometry proteomics offers a solution to this problem. Mass spectrometry (MS) represents one of the main technologies for quantitative proteomics with advantages and disadvantages. Quantitative MS has higher sensitivity but can provide only limited information about the intact protein. Quantitative MS has been used for both discovery and targeted proteomic analysis to understand global proteomic dynamics in populations of cells (bulk analysis) or in individual cells (single-cell analysis).

However, mass spectrometric methods introduce a number of other challenges, such as low sample preparation throughput and low protein identification rates without substantial instrument time. Proteolysis of complex biological samples can produce thousands of peptides, which may overwhelm the resolution capacity of known chromatographic and mass spectrometric systems, causing incomplete separation and impaired identification of the constituent peptides. Furthermore, performing large-scale plasma proteome profiling is challenging due to limitations imposed by lengthy preparation and instrument time. Throughput, reproducibility, time, and cost remain longstanding barriers to the necessary large-scale MS sample processing. There is a need to develop an accurate, reproducible, cost-effective, and time-effective method of which allows for identification and quantification of the various different peptides, polypeptides, and proteins.

### SUMMARY

Disclosed herein are methods of quantifying or peptides or polypeptides, comprising the steps of: (a) combining the peptides or polypeptides into one plex; (b) splitting the plex of peptides or polypeptides into two or more portions; (c) re-combining the portions into one plex; and (d) analyzing the peptides or polypeptides so as to obtain mass spectrometry data, thereby quantifying or identifying each peptide or polypeptide.

Also disclosed herein are methods of quantifying or identifying two or more chemically isotopic labeled peptides or polypeptides, comprising the steps of: (a) combining the two or more peptides or polypeptides into one plex; (b) splitting the plex of labeled peptides or polypeptides into two or more portions; (c) re-combining the portions into one plex; and (d) analyzing the peptides or polypeptides so as to obtain mass spectrometry data, thereby quantifying or identifying each peptide or polypeptide.

Also disclosed herein are methods of quantifying or identifying peptides or polypeptides, comprising the steps of: (a) combining the peptides or polypeptides into one plex; (b) mixing the peptides or polypeptides in the plex; (c) splitting the plex of peptides or polypeptides into two or more portions; (d) re-combining the portions into one plex; and (e) analyzing the peptides or polypeptides so as to obtain mass spectrometry data, thereby quantifying or identifying each peptide or polypeptide.

Also disclosed herein are methods of quantifying or identifying two or more chemically isotopic labeled peptides or polypeptides, comprising the steps of: (a) combining the two or more labelled peptides or polypeptides into one plex; (b) mixing the two or more labeled peptides or polypeptides in the plex; (c) splitting the plex of labeled peptides or polypeptides into two or more portions; (d) re-combining the portions into one plex; and (e) analyzing the peptides or polypeptides so as to obtain mass spectrometry data, thereby quantifying or identifying each peptide or polypeptide.

In some embodiments, the peptides or polypeptides are labeled. In some embodiments, the peptides or polypeptides are chemically isotopic labeled.

In some embodiments, the method further comprises mixing two or more peptides or polypeptides in a single plex prior to splitting a plex of peptides or polypeptides into two or more portions.

In some embodiments, the method further comprises clean-up of each portion. In some embodiments, the method further comprises clean-up of each portion prior to prior to splitting a plex of peptides or polypeptides into two or more portions. In some embodiments, the clean-up method is selected from the group consisting of peptide precipitation, in-solution (IS, in-StageTip (IST), Single-Pot Solid-phase enhanced Sample Preparation (SP3), filter-aided sample preparation (FASP), S-Trap, or SepPak.

In some embodiments, the method further comprises normalizing the concentration values of the peptides or polypeptide to a reference sample. In some embodiments, the normalizing is prior to labeling the peptides or polypeptides. In some embodiments, the method further comprises normalizing the concentration values of the peptides or polypeptides to a reference sample prior combining the peptides or polypeptides into one plex.

In some embodiments, the method further comprises generating a bridge.

In some embodiments, the analyzing comprises protein identification using high-field asymmetric waveform ion mobility (FAIMS Pro) and Real Time Search (RTS).

In some embodiments, the chemically isotopic labeled peptides or polypeptides are isobarically labeled or otherwise isotope incorporated.

In some embodiments, the method is performed on an automated liquid handling robot.

Also disclosed herein are methods for quantifying or identifying two or more chemically isotopic labeled peptides or polypeptides, comprising the steps of: (a) Combining the two or more labelled peptides or polypeptides into one plex; (b) Mixing the two or more labeled peptides or polypeptides in the plex; (c) Splitting the plex of labeled peptides or polypeptides into two or more portions; (d) Combining the portions into one plex; (e) Analyzing the peptides or polypeptides so as to obtain mass spectrometry data, thereby quantifying or identifying each chemical isotopic labeled peptide or polypeptide.

In some embodiments, the method further comprises clean-up of each portion prior to splitting the plex of labeled peptides or polypeptides into two or more portions, wherein the clean-up method is selected from the group consisting of peptide precipitation, in-solution (IS, in-StageTip (IST), Single-Pot Solid-phase enhanced Sample Preparation (SP3), filter-aided sample preparation (FASP), S-Trap, or SepPak.

In some embodiments, the method further comprises normalizing the concentration values of the two or more peptides or polypeptide to a reference sample prior to labeling the peptides or polypeptides.

In some embodiments, the method further comprises normalizing the concentration values of the two or more peptides or polypeptides to a reference sample prior to combining the two or more labelled peptides or polypeptides into one plex.

In some embodiments, the method further comprises generating a bridge.

In some embodiments, the method further comprises correlating the mass spectrometry data to each peptide or polypeptide.

In some embodiments, the chemical isotopic labeled peptides or polypeptides are isobarically labeled or otherwise isotope incorporated.

In some embodiments, the method is performed on an automated liquid handling robot.

Also disclosed herein are methods for obtaining a plurality of enriched peptides or polypeptides in a sample, comprising the steps of: (a) Contacting the sample with beads comprised of a single type of bead; (b) Separating a portion of the sample comprising protein-bound beads from a portion of the sample comprising unbound proteins; and (c) Resuspending the portion of the sample comprising protein-bound beads, thereby obtaining a plurality of enriched peptides or polypeptides in the sample.

In some embodiments, the sample is whole blood, plasma, serum, urine, saliva, tears, spinal fluid, synovial fluid, cell lysate, tissue lysate, exosomes and individual cell organelles. In some embodiments, the sample is plasma.

In some embodiments, the beads are magnetic. In some embodiments, the size of the beads is about 0.1 μm to 10 μm size in nanometers or 1 nm to 1000 μm size micrometers in diameter.

In some embodiments, the method further comprises incubating the beads with the sample at physiological conditions. In some embodiments the method is performed under physiological conditions. In some embodiments, the method is performed at a pH of about 7.4. In some embodiments, the method is performed at a temperature of about 37 degrees Celsius.

In some embodiments, the plurality of enriched peptides or polypeptides comprises one or more protein complexes.

In some embodiments, the method comprises magnetic immobilization to separate a portion of protein-bound beads from a portion of unbound plasma proteins and unbound beads in the sample.

In some embodiments, the method further comprises reducing the plurality of enriched peptides or polypeptides. In some embodiments, the method further comprises alkylating the plurality of enriched peptides or polypeptides. In some embodiments, the method further comprises derivatization of cysteines in the plurality of enriched peptides or polypeptides. In some embodiments, the method further comprises clean-up of the plurality of enriched peptides or polypeptides.

In some embodiments, the method comprises resuspending which includes digesting.

In some embodiments, the method further comprises digesting the plurality of enriched peptides or polypeptides. In some embodiments, the digesting comprises adding protease to the plurality of enriched peptides or polypeptides.

In some embodiments, the method further comprises labeling the plurality of enriched peptides or polypeptides. In some embodiments, the method further comprises isobarically labeling the plurality of enriched peptides or polypeptides. In some embodiments, the labeling comprises incorporating chemically isotopic labels onto each peptide or polypeptide.

In some embodiments, the method further comprises normalizing the concentration values of the two or more peptides or polypeptide to a reference sample prior to labeling the peptides or polypeptides.

Also disclosed herein are methods for quantifying or identifying peptides or polypeptides, comprising the steps of: (a) Obtaining a plurality of labeled peptides or polypeptides according to a method disclosed herein; (b) Aliquoting the labeled peptides or polypeptides into two or more portions; (c) Combining the two or more labeled portions into one plex; (d) Splitting the plex of labeled peptides or polypeptides into two or more portions for clean-up; (e) Re-combining the portions post clean-up; and (f) Analyzing the peptides or polypeptides so as to obtain mass spectrometry data, thereby quantifying or identifying peptide or polypeptide.

In some embodiments, the method further comprises mixing the two or more portions in the single plex prior to the splitting. In some embodiments, the method further comprises clean-up of each portion prior to re-combining.

In some embodiments, the method further comprises generating a bridge.

In some embodiments, the method is performed on an automated liquid handling robot.

Also disclosed herein are methods for quantifying or identifying peptides or polypeptides in a sample, comprising the steps of: (a) Contacting the sample with beads comprising a single type of bead; (b) Separating a portion of the sample comprising protein-bound beads from a portion of the sample comprising unbound proteins; (c) Resuspending the portion of the sample comprising protein-bound beads; (d) Incorporating isotopic labels onto each peptide or polypeptide in the portion of the sample comprising protein-bound beads; (e) Aliquoting the labeled peptides or polypeptides into two or more portions; (f) Combining the two or more isotopically labeled samples into one plex; (g) Splitting the plex of labeled peptides or polypeptides into two or more portions for clean-up; (h) Re-combining the portions into a single portion; and (i) Analyzing the peptides or polypeptides so as to obtain mass spectrometry data, thereby quantifying or identifying peptides or polypeptides in a sample.

In some embodiments, the method further comprises alkylating the portion of the sample comprising protein-bound beads. In some embodiments, the method further comprises clean-up of the portion of the sample comprising protein-bound beads. In some embodiments, the method further comprises the clean-up is prior to re-combining the portions into a single portion.

In some embodiments, the resuspending includes digesting. In some embodiments, the method further comprises digesting the peptides or polypeptides. In some embodiments, the digesting comprises adding protease to the plurality of enriched peptides or polypeptides.

In some embodiments, the method further comprises generating a bridge.

In some embodiments, the method is performed on an automated liquid handling robot.

Also disclosed herein is a method for quantifying or identifying peptides or polypeptides in a sample, comprising the steps of: (a) Contacting the sample with a single type of bead; (b) Separating a portion of the sample comprising protein-bound beads from a portion of the sample comprising unbound proteins; (c) Resuspending the portion of the sample comprising protein-bound beads; and (d) Analyzing the peptides or polypeptides so as to obtain mass spectrometry data, thereby quantifying or identifying peptides or polypeptides in a sample.

In some embodiments, the method further comprises alkylating the portion of the sample comprising protein-bound beads. In some embodiments, the method further comprises clean-up of the portion of the sample comprising protein-bound beads.

In some embodiments, the clean is prior to re-combining the portions into a single portion. In some embodiments, the resuspending includes digesting. In some embodiments, the method further comprises digesting the peptides or polypeptides. In some embodiments, the digesting comprises adding protease to the plurality of enriched peptides or polypeptides.

In some embodiments, the method further comprises generating a bridge.

In some embodiment, the method is performed on an automated liquid handling robot.

### BRIEF DESCRIPTION OF THE DRAWINGS

FIG. 1 is a graphical representation of a workflow outline for multiplexed proteomics.

FIG. 2A-2B shows instrument time requirements of label-free versus TMT 10-plex and TMTpro 16-plex across a range of samples, and a graphical representation of a workflow outline for multiplexed proteomics. FIG. 2A represents instrument time requirements to analyze a given number of samples with either label-free, TMT 10-plex, or TMTpro 16-plex across a range of samples. Estimates for TMT/pro were based on a three-hour run time plus a one-hour blank between samples. Estimates for label-free analysis were based on a one hour run time plus one-hour blank between samples. The number of injections includes the number of blanks injected. The number of TMTpro plexes was calculated assuming ‘15 samples plus bridge’ per injection. Similarly, TMT 10-plex assumes ‘9 samples plus bridge’. FIG. 2B is a graphical representation of sample preparation workflow.

FIG. 3A-3E is an evaluation of peptide recovery, digestion efficiency, and identification rates for sample preparation methods. FIG. 3A is a bar graph showing an initial comparison of methods by BCA assay measurements of peptide recoveries, expressed as %, using 100 μg as starting material for both mouse plasma (n=3) and cell lysate (n=6, error bars=standard deviation). IS=In-solution, MC=Methanol/Chloroform precipitation, iST=in-StageTip Preomics kit, SP3=Single pot solid phase enhanced sample preparation, ST (u/m)=S-Trap (micro/mini Protifi kits). FIG. 3B is a bar graph showing a comparison of identified total proteins, peptides and unique peptides across the five methods using cell lysate replicates (n=6, error bars=standard deviation). Performed using 2nd iST iteration, and several peptide recovery optimization iterations forward for SP3. FIG. 3C is a bar graph showing a comparison of identified total proteins, peptides and unique peptides across the five methods using mouse plasma replicates (n=3, error bars=standard deviation). Performed using 2nd iST iteration, and several peptide recovery optimization iterations forward for SP3. FIG. 3D is a bar graph showing digestion efficiency single shot comparison for mouse plasma replicates (n=3, error bars=standard deviation). Performed using 2nd iST iteration, and several peptide recovery optimization iterations forward for SP3. FIG. 3E is a graphical representation of considerations for committing to a method for automation between the ones evaluated. Methods evaluated and their ranking for practical aspects of large-scale sample preparation. Per category considered, with MC as reference method, ‘+’ indicates least favorable to ‘+++’ most favorable. Value assignment was either calculated (‘Cost’, ‘Time’), and/or based on hands-on experience and available tools/equipment (‘96-Well’, ‘Automation’, ‘Time’).

FIG. 4A-4B shows optimization of SP3 workflow and digestion conditions. FIG. 4A is bar graph showing SP3 peptide recovery optimization iterations using mouse plasma and cell lysate, with 100 μg starting material in each case (n=6, error bars=standard deviation). FIG. 4B shows mouse plasma total peptides (blue dots) versus mean missed cleavage (orange dots) comparison across various digestion optimization conditions, with peptide clean up performed on a 96-well Corning plate (n=3, error bars=standard deviation). DOC=Deoxycholate, TFE=Trifluoroethanol, Standard=50 mM HEPES pH 8.5. Both ‘1% DOC’ as well as ‘5% TFE’ conditions included ‘Standard’ buffer. R=Rapigest, P=PPS Silent Surfactant, I=Invitrosol.

FIG. 5A-5C shows label-free Comparison of SP3 and iST with technical mouse plasma injections, and TMT11 comparison of optimized MP3 and MC using mouse plasma injections FIG. 5A is a bar graph for data filtered at 1% FDR protein level and reverse hits removed (n=6). CV=Coefficient of Variation. FIG. 5B is a line graph showing evaluation of MP3 versus MC using TMT-labelled mouse plasma, single injections. Labeling efficiency (%), and digestion efficiency (%) reported with filtering at 1% FDR protein level, and reverse hits removed. Quantified total peptides, unique peptides, and total proteins, shown in table, filtered using the following criteria: sum signal-to-noise of 200, isolation specificity of 0.75. Quantified unique peptides used for CV plot: MP3=1276, MC=1685.

FIG. 6A-6E shows the development of an Automated Multiplexed Proteome Profiling Platform (AutoMP3) demonstrated on plasma. FIG. 6A is a graphical representation of development of an automated multiplexed proteome profiling platform (AutoMP3) demonstrated on plasma. Top candidate methods (iST and SP3) were evaluated and their ranking for practical aspects of large-scale sample preparation. Per category considered, with MC as reference method, ‘+’ indicates least favorable to ‘+++’ most favorable. Value assignment was either calculated (‘Cost’, ‘Time’) or based on hands-on experience and available tools/equipment (‘96-Well’, ‘Automation’, ‘Time’). FIG. 6B is a bar graph showing the results of a human-yeast spike-in experiment. Samples comprising 100 μg of human cell lysate were spiked with different amounts of yeast lysate: 0, 1, 2, 4 μg. Resulting accuracy of measurements of yeast peptides (summed signal-to-noise for TMT intensity) were compared to theoretical ratios. Error bars represent standard deviation (n=3), except for the no spike-in case (n=2). Total peptides and unique peptides quantified for both human and yeast are shown per method. FIG. 6C is a line graph showing per method coefficient of variation (CV) distributions of human peptides in the spike-in experiment. Filtering criteria include protein level FDR of 1%, sum signal-to-noise of 200, isolation specificity of 0.75, and reverse hits were removed. FIG. 6D is a graphical representation of MP3 automation on Hamilton Vantage™: deck layout usage across the entire process. Unused deck space not shown. CPAC=Cold Plate Air Cooled. FIG. 6E is a graphical representation of MP3 combine-mix-split strategy for cleanup of labelled TMT peptides. Immediately post TMT labeling, approximately 80 μL of aqueous volume is present per well in 200 μL 96-well plate (left panel). Each sample in the given plex is then pooled together and mixed in a well in a 2 mL 96-well plate (center panel). This amounts to 1280 μL aqueous volume, which cannot reach 95% organic content to bind peptides to magnetic beads for cleanup in a 2 mL 96-well plate-unless the combined plex is split back out into 16 portions of 80 μL aqueous volume and desalted separately (right panel). FIG. 6F is a bar graph showing the estimated time, in days, needed for AutoMP3 versus manual MC to prepare 16 and 96 TMT-labelled plasma samples, HPRP fractionation were not included. FIG. 6G is a table comparing manual-MP3 versus AutoMP3 using mouse plasma. ‘Peptide Recovery’ values quantified using BCA assay (n=24). TMT single shot results utilized a TMTpro 16-plex per method. ‘Average Ratio’, ‘Standard Deviation’, and ‘Coefficient of Variation’ were calculated using ratio of sum signal-to-noise of all sixteen channels. TMT results filtered to protein level FDR of 1%, >=200 sum signal-to-noise, >=0.75 isolation specificity, and reverse hits were removed.

FIG. 7A-E shows evaluation of robustness and TMT combine-mix-split strategy of AutoMP3 on Hamilton Vantage™. FIG. 7A is a bar graph showing ratiocheck evaluation of summed signal to noise of TMT channels prior to combine-mix-split strategy (left, CV=5.99%), and post (right, CV=5.09%). FIG. 7B shows the pattern with 100 μg starting protein material and indicated eight samples randomly selected for missed cleavage evaluation by single shot MS2 analysis on Orbitrap Lumos Fusion. FIG. 7C shows peptide recovery per well including per row/column variability. Expressed as % Coefficient of Variation (CV). FIG. 7D shows missed cleavage rates per tested well (expressed as % of total identified peptides). FIG. 7E shows summary statistics of peptide recovery (BCA assay-quantified, n=48), digestion efficiency, and protein and peptide identifications acquired on Orbitrap Lumos Fusion (n=8), filtered at 1% protein level FDR.

FIG. 8A-8B shows estimated time improvements automating MP3, and instrument analysis time comparison between label-free and TMTpro for various numbers of samples. FIG. 8A is a bar graph showing time savings (in hours) per step, going from manual MP3 (prepared in tubes, 2 plexes at a time) to AutoMP3 (prepared in 96-well plate, 6 plexes at a time), when preparing 96 plasma samples. FIG. 8B shows approximate instrument time needed to acquire DDA data for 16, 96, 960, and 9,600 samples using label-free or TMTpro 16plex (single shot or three fractions) sample preparation methods.

FIG. 9A-9B shows development of LC-MS method for robust and deep quantification of single-shot plasma samples. FIG. 9A is a bar graph and table representing method optimization using FAIMS Pro and RTS (SSN=Sum Signal to Noise). FIG. 9B is a protein abundance plot showing increased dynamic range with FAIMS Pro and RTS. Protein rank/abundance were derived by mapping identified gene names and/or protein accessions to the mouse plasma dataset at PAXdb (pax-db.org/dataset/10090/266/), with some highlighted lower abundance proteins detected (shown in the sub-table on the right).

FIG. 10A-10B shows evaluation of LC-MS method for robust identification of the same peptides across many TMTpro multiplexes. FIG. 10A shows evaluation of data dependent acquisition compared to using only an inclusion list. Peptide overlap in single mouse 16-plex across 5 injections. FIG. 10B is a bar graph showing evaluation of data dependent acquisition compared to using only an inclusion list. Peptide overlap in single mouse 16-plex across 15 injections.

FIG. 11A-11C shows evaluation of single shot versus three-part fractionation using MP3 FIG. 11A represents the experiment design of eluting peptides off magnetic beads using indicated elution solutions versus single solution elution (5% acetonitrile (ACN)) three injections. FIG. 11B shows the combined data results of given fractionated sets (‘1’, ‘2’, ‘3’, ‘4’) or unfractionated set ‘5’. Unfractionated set ‘5’, on average, resulted in 5% less total identified peptides, 29.5% less unique peptides, and 26.7% less total proteins (n=2). Contribution of total, unique peptides and total proteins per fraction for each of the five sets is indicated by colors blue (fraction 1), green (fraction 2), and orange (fraction 3). Additionally, identifications which overlap in 2 or more fractions are indicated in gray. FIG. 11C are venn diagrams of Unique Peptides and Total Protein contributions per each of the four fractionated sets and the fifth unfractionated set (n=2).

FIG. 12A-12B show svaluation of New Objective (25 cm), Aurora (25 cm, IonOpticks) columns, and uPAC chip (50 cm, Pharmafluidics) using both label-free HeLa and TMT16-labeled mouse plasma digests. FIG. 12A is a bar graph comparison of total peptides, unique peptides, and total proteins identified for label-free HeLa cell digest, as well as TMTpro 16-plex technical replicate mouse plasma digest injections. Filtered at 1% peptide level FDR (n=2, error bars=standard deviation). FIG. 12B is a bar graph showing median peak width elution times of total peptides for 1, 0.5, and 0.1 μg injections, filtered at peptide level FDR of 1% (n=2, error=standard deviation).

FIG. 13A-13D is a study design exploring circadian rhythm protein changes in naked mole-rat. FIG. 13A is a graphical representation showing bridge is generated using a small amount of material pooled from each of the samples (biological and technical) in the four plexes and labeled as 16th sample per 16-plex. First three timepoints for day 1 and day 2 had an additional technical replicate (both male and female), not shown, to have four complete 16-plexes. AutoMP3 sample preparation process took a total of three days (40 hours, no offline HPRP fractionation), with another day (16 hours) for Orbitrap Eclipse mass spectrometer data acquisition using FAIMS Pro & RTS. FIG. 13B is a table showing the number of identified and quantified total peptides, unique peptides, and total proteins for female and male plexes. Identified data filtered at 1% FDR protein level, reverse hits removed. FIG. 13C is a power analysis simulation using a 2-day experiment design and varying both the true amplitude of sinusoidal curves and the number of replicates per time point. FIG. 13D. shows examples of proteins without a clear circadian rhythm (Fgg: p-value=9.60e-1 FDR=9.73e-1, Hp: p-value-6.05e-1 FDR=8.23e-1, Itih4: p-value=9.13e-1 FDR=9.68e-1, Apoa4: p-value=1.04e-1 FDR=3.74e-1). The size of the circles represents magnitude of 1/Coefficient of Variation (CV) for the peptide measurements contributing to the given protein group for a biological replicate of a condition (time point). Largest circles indicate a 1/CV of >=160, and smallest circles indicate a 1/CV of <=160/3.

FIG. 14A-FIG. 14D a study design exploring proteome changes in response to UV irradiation in naked mole-rats and mice. FIG. 14A is a graphical representation showing a bridge is generated using a small amount of material pooled from each of the samples (biological and technical) in the same specie plexes and labeled as 16th sample per 16-plex. The control timepoint had additional technical replicates for both mouse and naked mole-rat, not shown, to reach six full 16-plexes. AutoMP3 sample preparation took a total of three days (40 hours, no offline HPRP fractionation), with another day (21 hours) for Orbitrap Eclipse mass spectrometer data acquisition using FAIMS Pro & RTS. FIG. 14B is a table showing identified and quantified total peptides, unique peptides and proteins for mouse and naked mole-rat experiments across the three plexes respectively. Identified data filtered at 1% FDR on the protein level, reverse hits removed. Quantified data additionally filtered to sum signal to noise of 200. FIG. 14C is a graph showing differential expression of protein changes in mouse and naked mole-rats between the control time point at 24-hours and 48 hours to 1-week post UV exposure. FIG. 14D is a comparison of quadratic time effects for selected proteins (Fgg: p-value=1.68e-4 FDR=2.29e-3, Hp: p-value=5.24e-3 FDR=2.90e-2, Itih4: p-value=3.44e-26 FDR-5.75e-24, Apoa4: p-value=1.38e-3, FDR=1.10e-2) between mouse and naked mole-rats across control 24 hour time point (0), and times post UV exposure from 30 minutes up to 168 hours (1 week). Size of circles represent magnitude as in FIG. 13D.

FIG. 15A-15B shows a heatmap and selected examples demonstrating how six-month UV treatment in mice dominates detected changes across experimental conditions. FIG. 15A shows a heatmap of three TMTpro mouse UV exposure plexes with control, and UV 30 minute through 6-month post UV exposure timepoints. Out of total quantified proteins, those with a fold change greater than two-fold are included in the heatmap (99/489). Colors determined by the centered-log-ratio of each estimated proportion. FIG. 16B shows proportion-space estimated values for protein-contributing-peptides per highlighted protein in mice (C8g, Igh-3, Itih4, Serpina7, Egfr, Serpina3k).

FIG. 16A-16B shows mouse and naked mole-rat changes up to one week after UV exposure. FIG. 16A shows additional protein response comparisons between mouse and naked mole-rat associated with Acute Protein Response pathway across 168 hours post UV exposure and control (time 0). Size of circles represent magnitude as in FIG. 13D. FIG. 16B shows mouse-only detected proteins.

FIG. 17 shows a graphical representation of a Native-MP3 workflow.

FIG. 18 is a Venn diagram of the total proteins identified using Native-MP3 coupled with transition to label-free mass spectrometry protocols with either 0.5 μm (right) or 1 μm carboxylate-modified (left), solid core magnetic particles.

FIG. 19 is a Venn diagram of the total proteins identified using Regular-MP3 (right) or Native-MP3 and coupled with transition to label-free mass spectrometry protocols with 1 μm carboxylate-modified, solid core magnetic particles (left).

FIG. 20 shows a graphical representation of the TMT multiplexed experimental design for the comparison of Native-MP3 and Regular-MP3 methodologies using plasma samples from WT and LPR/FAS mutant mice.

FIG. 21. is a Venn diagram of the total proteins quantified using Native-MP3 (left) and Regular-MP3 (right) after TMT labelling, 200 sum-signal-to-noise filter, reverse hits and contaminants removed.

FIG. 22A-22B are volcano plots of 1/(Coefficient of Variation) versus log 2 fold change showing the number of differentially expressed protein changes between WT versus FAS mice for Native-MP3 (FIG. 22A) and for Regular-MP3 (FIG. 22B). P_Null indicates the likelihood the detected fold change per protein was equal to or greater than two-fold.

FIGS. 23A-23C show the patterns and fold changes for exemplary proteins CD5L (FIG. 23A), Igh-1a (FIG. 23B), and Ighm (FIG. 23C) detected in both Native-MP3 and Regular-MP3.

FIG. 24 shows a comparison of quantified protein expression of proteins having greater than 2-fold protein fold changes for WT vs mutant (FAS) mice for Native-MP3 and Regular-MP3.

FIG. 25 shows a comparison of quantified protein expression of proteins having greater than 40% changes for WT vs mutant (FAS) mice for Native-MP3 and Regular-MP3.

FIG. 26 shows a comparison of quantified protein expression of all overlapping quantified proteins for WT vs mutant (FAS) mice for Native-MP3 and Regular-MP3.

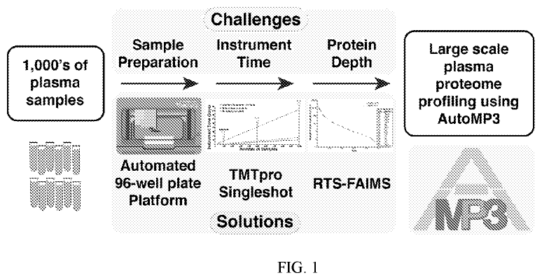

### DETAILED DESCRIPTION

The features and other details of the disclosure will now be more particularly described. Before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate particular embodiments of the invention, and not to exhaustively specify all permutations, combination and variations thereof.

Unless the context indicates otherwise, it is specifically intended that the various features described herein can be used in any combination. Moreover in some embodiments, any feature or combination of features set forth herein can be excluded or omitted. To illustrate, if the specification states that a complex comprises components A, B, and C, it is specifically intended that any of A, B, or C, or a combination thereof, can be omitted and disclaimed singularly or in any combination.

### Definitions

As used in the present specification, the following words and phrases are generally intended to have the meanings as set forth below, except to the extent that the context in which they are used indicates otherwise.

It is to be noted that as used herein and in the claims, the singular forms “a,” “an” and “the” include plural referents unless the context clearly dictates otherwise.

As used herein, “and/or” refer to and encompasses any and all possible combinations of one or more of the associated listed items, as well as the lack of combinations when interpreted in the alternative (“or”). Moreover, any feature or combination of features set forth herein can be excluded or omitted.

The term “about” as used herein when referring to a measurable value such as an amount of a compound or agent, dose, time, temperature, and the like, is meant to encompass variations of ±10%, ±5%, ±1%, ±0.5%, or even ±0.1% of the specified amount.

Unless otherwise defined, all technical and scientific terms used herein have the same meaning as commonly understood by one of ordinary skill in the art. The terminology used in the description herein is for the purpose of describing particular embodiments only and is not intended to be limiting.

As used herein, the term “sample” refers to mean any fluid, cell, tissue, organ or a portion thereof. It also includes, but is not limited to, whole blood, plasma, serum, urine, saliva, tears, spinal fluid, synovial fluid, cell lysate, tissue lysate, exosomes and individual cell organelles. The sample can be from any organism. It includes, but is not limited to human, mouse, yeast, worm, fish.

The term “peptide” refers to a short polymer of amino acids linked by peptide bonds. It has the same chemical (peptide) bonds as proteins but is commonly shorter in length. The shortest peptide is a “dipetide” consisting of two amino acids joined by a peptide bond. There can also be tripeptides, tetrapeptides, pentapeptides etc. A peptide has an amino end and a carboxyl end, unless it is a cyclic peptide.

The term “polypeptide” refers to a single linear chain of amino acids bonded together by peptide bonds and preferably comprises at least five amino acids. A polypeptide can be one chain or may be composed of more than one chains, which are held together by covalent bonds, e.g. disulphide bonds and/or non-covalent bonds. Polypeptides that can be purified with the method of the invention preferably have a length of at least five amino acids, more preferably a length of at least 10 amino acids, 15 amino acids, 20 amino acids, 25 amino acids, 30 amino acids, 35 amino acids, 40 amino acids, 45 amino acids or 50 amino acids or longer. In some embodiments, the peptides or polypeptides are suspended in a liquid solution. Examples of liquid solution include water, aqueous buffer mixtures, acidic or basic solutions, organic solvents such as alcohol or acetonitrile, or combination thereof.

The term “labeling” refers to a method of attaching a detectable moiety to polypeptides or introducing a detectably moiety into the polypeptides. Preferred are labels, markers or tags which provide quantification of labeled polypeptides in mass spectrometry analysis. The method of stable isotope labelling entails replacing specific atoms of the polypeptides, e.g. C, O, N, or S, with their isotopes. This includes but is not limited to methods like stable isotope labeling by amino acids in cell culture (SILAC), trypsin-catalyzed 180 labeling, isotope coded affinity tagging (ICAT), isobaric tags for relative and absolute quantitation (iTRAQ). One common method in the field of stable isotope labeling is dimethyl labeling comprising a reagent for the generation of a Schiff base with a primary amine, a reducing agent and a suitable buffer. Another widespread method is called isobaric labelling using tandem mass tag (TMT) labeling comprising a label reagent, a suitable buffer, an organic solvent, a denaturating reagent, a reducing reagent, an alkylating reagent, a quenching reagent and a protease.

The terms “splitting,” “separating,” or “portioning” and the like are used interchangeably herein to generally mean splitting one sample into a plurality of samples. The plurality of samples may have substantially equal volumes, the same volumes, or unequal volumes.

The term “plex” comprises isobaric labelsed peptides or polypeptides, isotopic labeled peptides or polypeptides, standard peptides or polypeptides, such as standard spike-in peptides or polypeptides, AQUA, PTM modified, non-PTM modified, noncanonical amino acids, unmodified peptides or polypeptides, unlabeled peptides or polypeptides, or any combination thereof.

The terms “enriched proteins”, “enriched polypeptides”, “enriched peptides” refers to proteins, polypeptides, or peptides that preferentially bind to and/or interact with a bead surface or a surface.

The term “physiological conditions” or “native” refers to conditions of the external or internal milieu that may occur in nature for an organism, cell, or cell system. Non-limiting examples of physiological conditions include a temperature range of 20-40 degrees Celsius, pH of 6-8, glucose concentration of 1-60 mM, or calcium concentration of 0.5-2 mM.

The term “non-physiological conditions” or “non-native” refers to conditions of the external or internal milieu that are in a higher or lower range than the conditions that may occur in nature for an organism, cell, or cell system. Non-limiting examples of non-physiological conditions include a temperature lower than 20 degrees Celsius, temperature higher than 40 degrees Celsius, pH less than 6, pH greater than 8, glucose concentration of less than 1 mM, glucose concentration greater than 60 mM, calcium concentration less than 0.5 mM, or calcium concentration greater than 2 mM.

### Large-Scale Plasma Proteome Profiling

Large-scale plasma proteome profiling typically requires a large time investment both in terms of sample preparation time and instrumental analysis. Non-limiting examples of samples includes whole blood, plasma, blood serum, red blood cells, white blood cells/PBMCs, cell lysate, tissue lysate, exosomes, samples enriched or sorted for specific cell types such as B cells, samples enriched for cell components such as organelles, samples enriched for PTMs (post-translational modifications), samples depleted or certain components such as high abundant proteins, samples enriched for certain proteins for example using immunoprecipitation.

The concentration of plasma proteins spans a dynamic range of at least 12 orders of magnitude, which results in poor protein identification depth. To combat the low number of proteins identified in plasma, the field has resorted to either extensive fractionation, or depletion of the most abundant proteins. Mass spectrometric analysis times with extensive fractions are expensive and are associated with a low rate of protein identification. While plasma depletion greatly increases the number of identifiable proteins, it is not amenable to automation, can lead to biases due to antibody cross-reactivity, and immunoprecipitation of antigen-bound proteins takes at least four hours to process. Furthermore, plasma depletion is costly and leads to increased variability.

Recent studies using multiplexed mass spectrometry-based proteomics analysis of human plasma quantified 652 proteins in depleted plasma in five hours of instrument time (10-plex quantification of approximately 131 proteins per hour) (Keshishian H, et al. 2017 Nat Protoc 12 (8): 1683-1701). From the same lab, depleted plasma combined with fractionation achieved 3,390 proteins with 4-plex quantitation (32 proteins per hour) (Keshishian H, et al. 2015 Mol Cell Proteomics 14 (9): 2375-2393). A recent study using undepleted plasma with 11-plex quantification, coupled to deep fractionation (180 fractions), achieved 4,826 proteins in approximately one month of instrument time (approximately 7 proteins per hour) (Dey K K, et al. 2019 Clinical Proteomics: 1-12). An alternative method is to use non-multiplexed quantitative proteomics (e.g. label-free or data-independent acquisition) with the largest undepleted dataset identifying 1,725 proteins at the time of this writing (Albrechtsen N J W, et al. 2018 Cell Systems 7 (6): 601-612.e3). However, non-multiplexed analyses require extensive instrument time and are sensitive to variations in liquid chromatography-mass spectrometry (LC-MS) performance (O'Connell J D, Paulo J A, O'Brien J J, Gygi S P 2018. J Proteome Res 17 (5): 1934-1942).

Nonlimiting examples of samples includes any fluid, cell, tissue, organ or a portion thereof. It also includes, but is not limited to, whole blood, plasma, serum, urine, saliva, tears, spinal fluid, synovial fluid, cell lysate, tissue lysate, exosomes and individual cell organelles. The sample can be from any organism. It includes, but is not limited to human, mouse, yeast, worm, fish. In some embodiments, the methods disclosed herein utilize a sample volume of about 0.5 μL-5000 μL. In some embodiments, the methods disclosed herein utilize a sample volume of about 50-250 μL. In some embodiments, the methods disclosed herein utilize a sample volume of about 250 μL.

### AutoMP3 Platform

Disclosed herein is an automated platform (also referred to as “AutoMP3”) for high throughput proteome profiling. The platform was designed considering and optimizing sample preparation, ease of automation, 96-well plate compatibility, level of multiplexing, LC column robustness and selection, LC-MS method acquisition parameters, fractionation, and how resulting outputs would feed into a downstream data analysis pipeline supporting statistical analysis and visualization of a large number of samples.

Most studies investigating the plasma proteome either use extensive fractionation for multiplexed samples, consequently requiring long instrument run times (1,025 proteins×1-plex/0.75 hr run/60 min=˜ 22 proteins/min/sample), or single shot runs using label-free quantification (652 proteins×10-plex/5 hr run/60 min=˜ 23 proteins/min/sample). A higher rate of proteins quantified in a short amount of time is needed for large scale biomarker studies that require the analysis of thousands of plasma samples. The AutoMP3 platform is able to quantify ˜44 proteins/min/sample (489 proteins×16-plex/3 hr run/60 min), an increase of approximately 2× over other known methods. The use of TMTpro combined with FAIMS Pro and RTS on an Eclipse mass spectrometer increased the rate of protein identification.

An increase in the number of quantified proteins in plasma by around 50% was achieved using the AutoMP3 Platform. This improvement was made possible largely by the use of FAIMS Pro and RTS. The nature of DDA methods means that different peptides are selected for sequencing between plexes, which inevitably decreases overlap and leads to missing values. To maximize overlap between plexes, an inclusion list method approach was investigated. For large numbers of plexes (>15), using an inclusion list method on its own is likely the preferred method to achieve the largest peptide overlap compared to DDA. However, for smaller numbers of plexes, the regular DDA method, in our setup, resulted in the largest coverage and peptide overlap between plexes. Another advantage of the inclusion list approach is the ability to prioritize peptides of interest. This is particularly attractive for low abundance PTM peptides that would not otherwise be selected for analysis.

The utility of automated scale fractionation was also evaluated. A potentially worthwhile increase in peptide and protein identifications was observed, which needs to be weighed against longer instrument time, especially for large scale experiments. Fractionation was easily implemented with the AutoMP3 platform using a modified version of the peptide cleanup protocol, where instead of eluting peptides with a single buffer, three (or more) elution buffers could be applied (for example, a gradient of high to low concentration of acetonitrile) and each transferred to separate 96-well plates. Fractionating using AutoMP3 in the 96-well format allowed for higher throughput than our standard high-pressure reverse phase (HPRP) fractionation setup. As with other steps, fractionation was designed in a modular fashion. The effect of fractionating was tested on label-free mouse plasma samples into three parts, using various acetonitrile-based elution gradients. Fractionated sets were compared against three single shot injections of an unfractionated sample (FIG. 11A). While within the various fractionation gradients a major difference of unique and total proteins was not identified, and the difference between fractionated and unfractionated sets was noticeable. An increase of 29.5% and 26.7% more unique peptides and total proteins identified was observed, respectively (FIG. 11B).

To minimize instrument acquisition time and the associated costs, sample multiplexing and throughput was maximized using TMTpro and single shot analyses. The main barrier to performing large-scale studies using TMT is sample preparation, since the labeling chemistry is very time consuming to perform manually. This challenge may be addressed by creating a magnetic bead-based, TMT-compatible/isobaric labeling sample preparation workflow.

### Protein Quantification

In some embodiments, the present disclosure provides methods comprising quantitating the glycosylated peptide fragments by using a mass spectrometer (MS). In some embodiments, the methods employ liquid chromatography (LC).

In some embodiments, the method comprises further fractionation prior to analyzing. In some embodiments, the method comprises further splitting of the peptides or polypeptides prior to analyzing. In some embodiments, the method comprises further splitting of the combination of peptides or polypeptides prior to analyzing.

Disclosed herein, in certain embodiments are methods for quantifying or identifying two or more chemically isotopic labeled peptides or polypeptides, comprising the steps of: (a) combining the two or more labelled peptides or polypeptides into one plex; (b) splitting the plex of labeled peptides or polypeptides into two or more portions; (c) combining the portions into one plex; and (d) analyzing the peptide or polypeptides so as to obtain mass spectrometry data, thereby quantifying or identifying each chemical isotopic labeled peptide or polypeptide.

Disclosed herein, in certain embodiments are methods for quantifying or identifying two or more chemically isotopic labeled peptides or polypeptides, comprising the steps of: (a) combining the two or more labelled peptides or polypeptides into one plex; (b) mixing the two or more labeled peptides or polypeptides in the plex; (c) splitting the plex of labeled peptides or polypeptides into two or more portions; (d) combining the portions into one plex; and (e) analyzing the peptides or polypeptides so as to obtain mass spectrometry data, thereby quantifying or identifying each chemical isotopic labeled peptide or polypeptide.

In some embodiments, the method further comprises mixing the two or more labeled peptides or polypeptides in the single plex prior to splitting.

In some embodiments, the method further comprises clean-up of each portion of sample or ple off samples prior to combining. In some embodiment, the clean-up is selected from the group consisting of peptide precipitation, in-solution (IS, in-StageTip (IST), Single-Pot Solid-phase enhanced Sample Preparation (SP3), filter-aided sample preparation (FASP), STRAP, or SEP PAK.

In some embodiments, the method further comprises normalizing the concentration values of the two or more peptides or polypeptide to a reference sample. In some embodiments, the method further comprises normalizing the concentration values of the two or more peptides or polypeptide to a reference sample prior to labeling the peptides or polypeptides.

In some embodiments, the method further comprises normalizing the concentration values of the two or more peptides or polypeptides to a reference sample prior to combining the two or more labelled peptides or polypeptide. In some embodiments, the method further comprises normalizing the concentration values of the two or more peptides or polypeptides to a reference sample prior to combining the two or more peptides or polypeptide.

In some embodiments, the method further comprises generating a bridge.

In some embodiments, the analyzing comprises protein identification. In some embodiments, the protein identification is using high-field asymmetric waveform ion mobility (FAIMS Pro) and Real Time Search (RTS).

In some embodiments, the chemically isotopic labeled peptides or polypeptides are isobarically labeled or otherwise isotope incorporated.

In some embodiments, the method is performed on an automated device. In some embodiments, the method is performed on a liquid handling robot.

### TMT

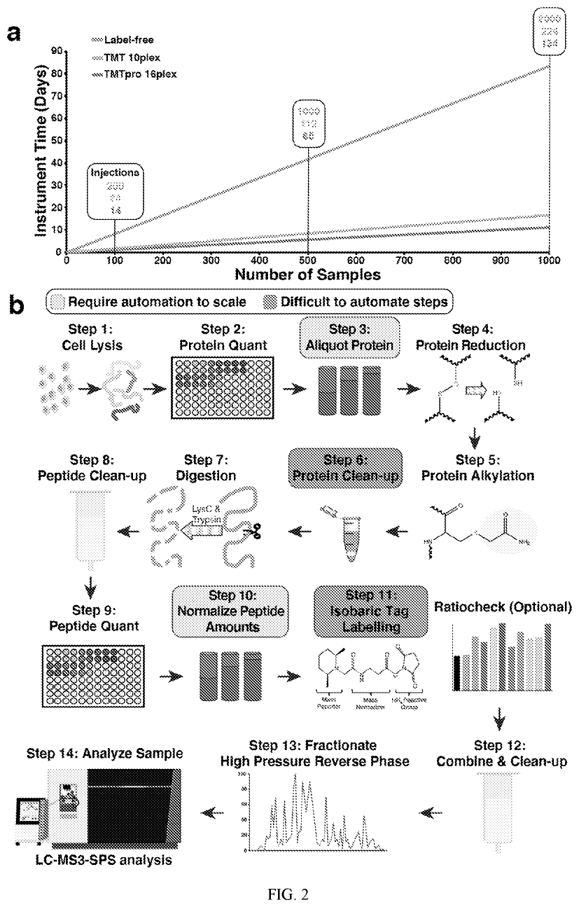

Minimizing instrument time, while maximizing the number of proteins quantified per sample, is critical to allow large cohort analysis at a reasonable cost. When using label-free mas spectrometry type approaches, the instrument time to sample ratio increases at a much greater rate than for multiplexed isobaric tag methods, such as TMT 10plex and TMTpro 16-plex (FIG. 2A).

For example, analyzing 1,000 samples would require three months of continuous LC-MS time using label-free, whereas TMT multiplexing would require 19 days and would require 12 days (FIG. 2A). Additionally, the TMTpro approach only requires 134 injections (including blanks), compared to 2,000 injections for the label-free approach. Furthermore, this reduction in injection translates to less degradation of the injection needle and liquid chromatography (LC) column. Thus, increasing the level of multiplexing while decreasing run time reduces the cost of analyzing large sample cohorts. However, the standard TMT sample preparation protocol requires many more steps than a label-free protocol.

Disclosed herein are methods of automated sample preparation solution for TMTpro. As depicted in FIG. 2B, an exemplary sample preparation workflow for isobaric labeling with TMT involves multiple steps. Following lysis, the protein amount in each sample is quantified, aliquoted, and normalized to the same concentration. The proteins are then reduced and alkylated. The sample is cleaned up prior to enzymatic digestion. Non-limiting examples of enzymatic digestion include digestion with LysC, digestion with trypsin, and digestion with LysC and then with trypsin. The resulting peptides are subsequently cleaned up. The peptides are then quantified, and normalized amounts are labeled using TMT, and the reaction is quenched. Prior to combining, there is an optional ratio check step to ensure equal amounts of protein were labeled for each TMT channel. The labeled samples are combined and then cleaned up together. Following cleanup, the sample is then fractionated off-line by HPRP or is analyzed directly by LC-MS3.

### Labels

Labeling samples with stable isotope labels allows a mass spectrometer to distinguish between identical proteins in separate samples. Protein quantitation is accomplished by comparing the intensities of reporter ions in the MS/MS spectra. A key benefit of isobaric labeling over other quantification techniques (e.g. SILAC, ICAT, Label-free) is the increased multiplex capabilities and thus increased throughput potential. The ability to combine and analyze several samples simultaneously in one LC-MS run eliminates the need to analyze multiple data sets and eliminates run-to-run variation. Multiplexing reduces sample processing variability, improves specificity by quantifying the proteins from each condition simultaneously, and reduces turnaround time for multiple samples. Isobaric chemical tags can facilitate the simultaneous analysis of multiple samples.

In some embodiments, simultaneous analysis can be conducted for up to 100 samples, including but not limited to 2 samples, 6 samples, 10 samples, 11 samples, 16 samples, and 18 samples.

In some embodiments, the peptides or polypeptides are isotopically labeled. In some embodiments, protein or peptide samples prepared from cells, tissues or biological fluids are labeled in parallel with the isobaric mass tags and combined for analysis.

Non-limiting examples of labels for relative quantification methods include isobaric labeling (tandem mass tags or TMT), isobaric tags for relative and absolute quantification (iTRAQ), label-free quantification metal-coded tags (MeCAT), N-terminal labeling, stable isotope labeling with amino acids in cell culture (SILAC), terminal amine isotopic labeling of substrates (TAILS), and Isotope-coded affinity tag (ICAT).

Metal-coded tags (MeCAT) method is based on chemical labeling, but rather than using stable isotopes, different lanthanide ions in macrocyclic complexes are used. Stable isotope labeling with amino acids in cell culture (SILAC) is a method that involves metabolic incorporation of “heavy” C- or N-labeled amino acids into proteins followed by MS analysis. Traditionally the level of multiplexing in SILAC was limited due to the number of SILAC isotopes available.

Isobaric mass tags (tandem mass tags or TMT) are tags that have identical mass and chemical properties that allow heavy and light isotopologues to co-elute together. These tags are designed to cleave at a specific linker region upon high-energy CID, yielding different-sized tags that are then quantitated by LC-MS/MS.

In some embodiments, the two or more peptides or polypeptides, comprise one or more chemically labeled peptides or polypeptides. In some embodiments, the two or more peptides or polypeptides, comprise one or more unlabeled peptides or polypeptides. In some embodiments, the two or more peptides or polypeptides, comprise one or more labeled peptides or polypeptides and/or one or more unlabeled peptides or polypeptides. In some embodiments, the two or more peptides or polypeptides, comprise one or more chemically labeled peptides or polypeptides and/or one or more unlabeled peptides or polypeptides. In some embodiments, the two or more peptides or polypeptides, comprise one or more chemically labeled peptides or polypeptides and/or one or more unlabeled PTM peptides or polypeptides. In some embodiments, the methods comprise two or more labeled peptides or polypeptides, wherein the labels are not the same. In some embodiments, the methods comprise two or more labeled peptides or polypeptides, wherein the labels are the same.

### Beads

Magnetic bead type workflows are ideal for automation because they are magnetic and bead quantity is easily adjusted based on the starting amount. This is in contrast to solid phase systems, which are limited to cartridges with a set capacity.

In some embodiments, the beads are comprised of a single type of bead. In some embodiments, the beads are magnetic. In some embodiments, the beads are not magnetic.

In some embodiments, the size of a bead is less than 1 micron in diameter. In some embodiments, the size of a bead is about 1-10,000 size in nanometers. In some embodiments, the size of a bead is 0.1-1,000 size micrometers in diameter. Suitable examples of ranges of a bead size include, but are not limited to, for example, a bead from about 5 nm to about 1000 nm in diameter, about 5 nm to about 500 nm, about 10 nm to about 500 nm, about 50 nm to about 500 nm, alternatively about 50 nm to about 250 nm, alternatively about 50 nm to about 200 nm, alternatively about 50 nm to about 100 nm, and any combinations, ranges or amount in between (e.g., 10 nm, 20 nm, 30 nm, 40 nm, 50 nm, 60 nm, 70 nm, 80 nm, 90 nm, 100 nm, 150 nm, 200 nm, 250 nm, 300 nm, 350 nm, 400 nm, 450 nm, 500 nm, 550 nm, 600 nm, 650 nm, 700 nm, 750 nm, 800 nm, 850 nm, 900 nm, 1000 nm, etc.).

In some embodiments the beads are, for example, nanoparticles, microparticles, protein-based particles, and combinations thereof. In some embodiments the beads have a solid core. In some embodiments the beads have a porous core. In some embodiments the beads surface is functionalized. Examples of functional group include but are not limited to hydrophobic surfaces (e.g. C18) with carboxylate groups to provide hydrophilicity.

Non-limiting examples of beads include 1 μm average diameter hydrophobic surface carboxylate SpeedBead SeraMag beads (Cytiva, #44152105050350), 0.5 μm, carboxylate SpeedBead SeraMag beads, solid core, 5 μm, amine, porous core, 5 μm, carboxylated, porous core, 5 μm, HILIC, porous core, 5 μm, hydroxyl, porous core, 1 μm, carboxylated, solid core, or a combination thereof.

In some embodiments, a method disclosed herein comprises hydrophobic SeraMag SpeedBeads (Cytiva, #44152105050350). In some embodiments, a method disclosed herein comprises hydrophilic SeraMag SpeedBeads (Cytiva, #45152105050350). In some embodiments, a method disclosed herein comprises a mixture of hydrophobic and hydrophilic SeraMag SpeedBeads (Cytiva, #44152105050350 & #45152105050350). In some embodiments, a mixture of hydrophobic and hydrophilic SeraMag SpeedBeads is in a 1:1 ratio.

### Automation

Disclosed herein are methods of automated sample preparation. Automated sample preparation allows a 96-well plate automation solution, as well as a high degree of flexibility for future improvements (e.g., multiplexing reagents).

Multiple automated sample preparation platforms for label-free proteomics are known in the art. A non-limiting example of a commercially available robot that can handle chemical isotopic labeling includes the PreON workflow (Preomics, Planegg-Martinsried, Germany). However, the PreON only allows multiplexing of up to 11 samples simultaneously. Additionally, the PreON is not compatible with TMTpro, and it cannot multiple plexes because it can only process 12 samples in one round. The PreON system is capable of processing up to 36 samples per day, which then allows for three 11-plexes (10 samples+bridge). However, this solution is not compatible with 16-plex automation, does not include automated TMT label ratio check sample generation, does not include adjustments of samples with different protein concentrations (normalization), does not include bridge generation and it is not compatible with a 96-well plate format design.

In some embodiments, the method relates to an automated system with centralized control for performing proteomics research and sample preparation. In some embodiments, the automated system is a liquid handling. In some embodiments, the automated system is a pipetting robot. In some embodiments, the automated system is a microfluidic device.

In some embodiments, a method disclosed herein is performed on an automated device. In some embodiments, a method of quantifying or identifying is performed on an automated device. In some embodiments, a method of quantifying or identifying is performed on a a liquid handling robot. Examples of liquid handler platforms include but are not limited to Hamilton Vantage™, Tecan, Agilent Bravo, Opentrons OT-2, or epMotion 5073m. An example of a microfluidic device includes but is not limited to a digital microfluidic device (Sci-bots, DropBot DB3-120). In some embodiments, MP3 sample preparation may be automated on a Hamilton Vantage™ liquid handler.

While the most difficult and important steps in automating the manual protocol were the protein cleanup and TMT labeling, there were additional steps that were also critical to automate when scaling up from a single 16-plex to dozens and even hundreds of plexes (FIG. 2B). These steps included sample normalization, bridge generation, and ratio check. When performing these steps manually, a great deal of focus and patience is required to avoid any pipetting errors or mistakes. Automating these steps greatly reduces the chance for these “operator errors” and allows large numbers of plexes to be processed.

In addition to automating a pooled bridge design for combining data between plexes, an in-house software platform was developed for analyzing time courses based on previously described protein quantification models. This allows for processing large numbers of plexes together and fit linear, quadratic, or cubic time effects, while using the within-sample variability to weight each observation. This weighting prevents highly variable measurements from unduly influencing estimated trajectories.

### Sample Normalization

In some embodiments, a method disclosed herein further comprises normalizing concentration values of the two or more peptides or polypeptides to a reference sample prior to labeling the peptides or polypeptides. In some embodiments, the normalizing is by BCA.

In some embodiments, the method further comprises normalizing the concentration values of the two or more peptides or polypeptides to a reference sample prior to step (a). In some embodiments, the normalizing is by ratio check. In some embodiments, the ratio check normalizes peptide content in each channel to ensure equal total amounts of total peptide/protein in each sample using mass spectrometry readout of peptide concentration.

### Bridge Generation

A challenge in a TMT workflow arises when the number of experimental samples exceeds the number of TMT multiplex channels. Typically, this challenge is mitigated with a bridge channel that contains a mixture of all samples. Each individual experimental channel can be normalized to the bridge to enable combining multiple plexes together. To construct the bridge, a small portion of each sample at the peptide level may be combined prior to labeling.

In some embodiments, a method disclosed herein further comprises generating a bridge. In some embodiments, a bridge comprises a small portion of each sample. In some embodiments, a bridge is constructed by taking a portion of each sample and combining each portion into one. In some embodiments, a bridge comprises taking a portion of select samples and combining each portion into one. In some embodiments, a bridge comprises a mixture of synthetic peptides, a select set of samples, or a portion of each sample. In some embodiments, a bridge is constructed prior to labeling. In some embodiments, a bridge is generated prior to labeling the peptides or polypeptides.

### Ratio Check

A ratio check sample is generated by taking a portion of each samples (e.g. 0.1-20%), combined, and then performing clean-up prior to mass spectrometry analysis to obtain an estimate of peptide/protein abundance of each of the samples. A ratio check step allows for correction to ensure equal peptide/protein abundance of each of the samples. Non-limiting examples of methods of protein/peptide quantification includes colorimetric, fluorescent based assays such as the BCA or Bradford assay, and use of a ratio check sample to ensure substantially equal protein concentrations between samples.

In some embodiments, a method disclosed herein further comprises ratio check. In some embodiments, ratio check comprises normalizing peptide content in each channel to ensure equal peptide/protein concentrations in each sample prior to combining the samples for analysis by mass spectrometry.

### Clean-Up

In some embodiments, a method disclosed herein further comprises clean-up. In some embodiments, the clean-up method is selected from the group consisting of peptide precipitation, in-solution (IS), in-StageTip (IST), Single-Pot Solid-phase enhanced Sample Preparation (SP3), filter-aided sample preparation (FASP), S-TRAP, or C18 column based clean-up such as SepPak (Waters). In some embodiments, clean-up is by de-salting. In some embodiments, clean-up is by detergent removal. In some embodiments, clean-up is by lipid or metabolite removal. In some embodiments, clean-up is by small molecule removal.

In some embodiments, a method disclosed herein further comprises a peptide cleanup protocol. In some embodiments, a peptide cleanup protocol comprises eluting peptides with a single buffer. In some embodiments, a peptide cleanup protocol comprises more than one elution buffer. In some embodiments, a peptide cleanup protocol comprises three or more elution buffers. In some embodiments, in a peptide cleanup protocol comprising more than one elution buffer, a gradient of high to low concentration can be created. In some embodiments, a gradient of high to low concentration is of acetonitrile or other oganic solvent. In some embodiments, a gradient of low to high acetonitrile or other organic solvent. In some embodiments, a peptide cleanup protocol comprises transferring each elution to separate 96-well plates or single vessel consumables, or 384-well plates, or any number in between/higher etc.

In some embodiments, a peptide cleanup protocol is prior to combining the two or more labeled peptides or polypeptides. In some embodiments, a peptide cleanup protocol is prior to combining two or more portions into one plex. In some embodiments, a peptide cleanup protocol is prior to MS data acquisition analysis.

### Comparison of Performance

Nonlimiting examples of comparisons of performance include comparisons of: average number of peptide and protein identifications, missed cleavage rates, variability in method, labeling efficiency (%) MC vs iST vs MP3, quantified total peptides, unique peptides, and total proteins, accuracy and precision, based on the yeast spike-in peptides.

### EXAMPLES

The disclosure is further illustrated by the following examples. The examples are provided for illustrative purposes only, and are not to be construed as limiting the scope or content of the disclosure in any way.

### Example 1: Optimization and Development of the Manual Multiplexed Proteome Profiling Platform (MP3)

Four sample preparation methods were evaluated as alternatives to MC: in-solution (IS), in-StageTip (IST), Single-Pot Solid-phase enhanced Sample Preparation (SP3) and S-Trap (ST).

In some embodiments, clean-up is prior to combining the two or more label peptides or polypeptides. In some embodiments, clean-up is prior to combining the two or more portions into one plex. In some embodiments, the clean-up method is selected from the group consisting of peptide or protein precipitation, in-solution (IS), in-StageTip (IST), Single-Pot Solid-phase enhanced Sample Preparation (SP3), filter-aided sample preparation (FASP), S-TRAP, or C18 column based clean-up such as SepPak (Waters) In some embodiments, clean-up is by de-salting. In some embodiments, clean-up is by detergent removal. In some embodiments, clean-up is by lipid or metabolite removal. In some embodiments, clean-up is by small molecule removal.

Method Comparison and Optimization Samples. For all label-free method comparison studies as well as SP3 digestion optimization either cell lysate of A-375 melanoma cells or mouse plasma from naive C57/B6 mice were used. Mouse plasma was collected in-house from C57/B6 mice. For the yeast spike-in experiment to assess precision and accuracy of TMT labelled 11-plex, DBY12000 yeast strain and A-375 melanoma cells were used.

### Sample Preparation

Lysate Generation. Unless otherwise indicated, lysis buffer of 75 mM NaCl, 50 mM HEPES (4-(2-hydroxyethyl)-1-piperazineethanesulfonic acid) pH 8.5, 3% SDS (sodium dodecyl sulfate), with Roche Protease Inhibitor cocktail was added to A375 cell pellet or mouse plasma. Cell lysates were additionally sonicated with EpiShear sonicator probe (ActivMotif, Carlsbad, CA) (cycle settings: amplitude of 40%, pulse 10 seconds ON, 5 seconds OFF, for a total time of 50 seconds). Protein quantification was performed using the bicinchoninic acid (BCA) assay (Thermo Fisher Scientific, Waltham, MA). Proteins were reduced with dithiothreitol (DTT, 5 mM, 30 min, 250 C), alkylated with iodoacetamide (IAA, 15 mM, 30 min, 25° C., in the dark), and excess IAA was quenched with equivalent DTT (5 mM, 25° C., 5 min in the dark). An aliquot of 100 μg of protein was then purified using either methanol-chloroform protein precipitation (MC), In-solution (IS), in-StageTip (IST), Single-pot solid-phase enhanced sample preparation (SP3) or S-Trap (ST).

Vacuum Assisted Sample Desalting using SepPack columns. Column was first equilibrated with 1×1 mL 100% methanol. Followed by equilibration with 1×1 mL 80% acetonitrile (ACN). Followed by 1×1 mL wash of 0.5% acetic acid. Followed by 3×1 mL washes of 0.1% trifluoroacetic acid (TFA). Acidified sample was then bound to column resin. This was followed by another 3×1 mL wash of 0.1% TFA. Followed by 1×1 mL wash of 0.5% acetic acid. Cleaned up peptides were then eluted with 1×0.75 mL 40% ACN, 0.5% acetic acid, and 1×0.75 mL 80% ACN, 0.5% acetic acid. Elutions were combined and dried down using a speedvac.

Methanol/Chloroform Sample Preparation. Samples were methanol-chloroform precipitated as previously described (McAlister G C, et al. (2014) MultiNotch MS3 Enables Accurate, Sensitive, and Multiplexed Detection of Differential Expression across Cancer Cell Line Proteomes. Anal Chem 86 (14): 7150-7158). Briefly, to one volume of sample, four volumes of 100% methanol, one volume of 100% chloroform, and three volumes of 100% water were added with a vortex mix step after each addition, followed by a centrifugation at 15,000 g for 30 minutes at 4° C. to clarify the precipitated protein pellet, which was followed by removing both of the liquid phases, a 1000 μL 100% methanol wash and centrifugation at 15,000 g for 10 minutes at 4° C., removal of wash and air drying. The precipitated protein was resuspended in 100 μL 8M urea, 50 mM HEPES pH 8.5. The sample was then diluted to 4M urea and digested first with LysC at a ratio of 1:100 protease: protein (16 h, 25° C.). The samples were then diluted to 1 M urea and digested with trypsin at 1:25 protease: protein (6 h, 300 rpm, 37° C.). Samples were then acidified (final concentration 0.5% TFA. Samples were centrifuged to remove cellular debris, and supernatant saved (15,000 g, 10 min, 4° C.). Samples were then desalted using C18 solid-phase extraction (SPE) columns (SepPak) as described above and dried down in a speedvac overnight. Peptide BCA was then performed to quantify peptide content. (Thermo Fisher Scientific, Waltham, MA). For a label-free mass spectrometry experiment, samples were resuspended in 5% formic acid & 5% ACN (MS Loading Buffer), and 1 μg was injected on the instrument. For TMT experiments, 50 μg peptide material was then aliquoted per sample to prepare for labeling. For an 11-plex/16-plex TMT experiment a ratio of 4:1/8:1 of TMT reagent: peptide was used per sample for cell lysate and mouse plasma respectively. Labeling was performed in 50 μL 200 mM HEPES, 30% anhydrous ACN for 1 hour shaking at 300 rpm, and room temperature. Reaction was quenched with 5% hydroxylamine, 200 mM HEPES pH 8.5 to final concentration of 0.3% v/v for 15 minutes at room temperature. For label ratio check, 4 μL was taken out each labelled sample combined, desalted using a StageTip, and analyzed on the instrument. Using the label ratio check, samples were normalized, combined, and desalted using C18 SepPak, as described above. High pH Reversed-Phase (HPRP) fractionation, was performed as described below. For mass spectrometry analysis TMT-based fractionated experiments, 12 fractions of set A were each resuspended in 20 μL MS Loading Buffer and 2 μL per fraction were analyzed by Orbitrap Lumos Fusion mass spectrometer. For label-free unfractionated experiments, 10 μg per sample was resuspended in 20 μL MS Loading Buffer and 1 μg analyzed on the instrument.

In-Solution (IS). Both mouse plasma and cells were lysed as described above except with the following lysis buffer: 8 M Urea 50 mM HEPES pH 8.5, with Roche Protease Inhibitor cocktail. No cleanup post lysis and prior to digestion was performed. Digestion and peptide desalting were performed the same way as described in methanol/chloroform method. Samples were resuspended in MS Loading Buffer and a BCA assay was then performed to assess peptide material recovered. For label-free instrument analysis, 10 μg were resuspended in 20 μL MS Loading Buffer and 2 μL (1 μg) were analyzed by LC-MS/MS.

Label-free in-StageTip (IST) method. For initial method comparison the label-free iST method is used as per manufacturer's instructions (Preomics, Martinsried, Germany). In brief, approximately 100 μg mouse plasma or cell lysate samples were reduced and alkylated at 95° C. for 10 minutes, shaking at 1,000 rpm. BCA assay was performed to adjust sample amounts to be exactly 100 μg for the proceeding digestion step. Samples were digested over the filter cartridge for 3 hours at 37° C., 500 rpm. Digestion was quenched and samples were bound to filter by spinning at 3,800 g for 2 minutes. Samples were washed once with two sequential wash solutions, and then eluted twice with 100 μL of elution buffer. Samples were dried down in a speed-vac overnight, resuspended in 200 μL MS Loading Buffer at an approximate concentration of 0.5 μg/μL, and 2 μL (1 μg) was analyzed by LC-MS/MS.

TMT-compatible in-StageTip (iST-NHS) method. For yeast spike-in experiment the TMT-compatible iST method was used as described by the vendor in an earlier version where it was advised to perform TMT labeling on top of the filter. A more recent version advises to perform TMT labeling in-solution in a tube, and then transferring the labelled sample to the filter for wash—this produces better labeling efficiency. In brief, approximately 100 μg mouse plasma or cell lysate samples were lysed, reduced and alkylated using iST specific alkylating reagent (C6H11NO composition, 113.084 Da) in a TMT-compatible lysis buffer at 95° C. for 10 minutes, shaking at 1,000 rpm. BCA assay was performed to adjust sample amounts to be exactly 100 μg for the proceeding digestion step. Samples were digested over the filter cartridge for 3 hours at 37° C., 500 rpm. TMT reagent was added at a ratio of 8:1 for mouse plasma, and 4:1 for cell lysate and labeling was performed at room temperature, 500 rpm, for 2 hours. Labeling reaction was quenched with 12 μL 5% hydroxylamine solution for final concentration of 0.3% v/v. Digestion was quenched and samples were bound to filter by spinning at 3,800 g for 2 minutes. Samples were washed once with two sequential wash solutions, and then eluted twice with 100 μL of elution buffer. Samples were dried down in a speed-vac overnight, resuspended in 200 μL MS Loading Buffer (˜0.5 μg/μL) and 2 μL (1 μg) analyzed on the mass spectrometer for a ‘ratio check’. Samples were then combined based on ratio corrected sum signal to noise values per TMT channel and dried down. HPRP fractionation was performed as described below. For mass spectrometry analysis, 12 fractions of set A were each resuspended in 20 μL MS Loading Buffer per fraction and 2 μL were analyzed by Orbitrap Lumos Fusion mass spectrometer.

S-Trap (micro): range of <=50-100 μg & S-Trap (mini): range of 100-300 μg. Sample preparation was performed as indicated by the kit (Protifi, Huntington, NY). Epishear probe sonication method was performed as described above for cell lysis. Protein- and peptide-level quantification was performed using the BCA assay. A ratio of 1:25 of trypsin: protein was used to digest 100 μg of either mouse plasma or cell lysate material. For label-free instrument analysis, 10 μg were resuspended in 20 μL MS Loading Buffer and 2 μL (1 μg) were analyzed by LC-MS/MS.

Optimization of peptide recovery for SP3. For each trial 100 μg of starting protein material (human cell lysate and mouse plasma) was processed starting with lysis through digestion stage with and without peptide cleanup. Peptide level quantitation was performed using BCA assay in the digestion buffer or diluted elution buffer (to maintain compatibility with BCA assay reagents) utilized for the trial. Variations of digestion volumes, Sera-Mag magnetic beads: protein ratios, wash, elution buffers as well as conditions and switching to processing samples in PCR tubes up to peptide cleanup stage were performed to improve peptide recovery.

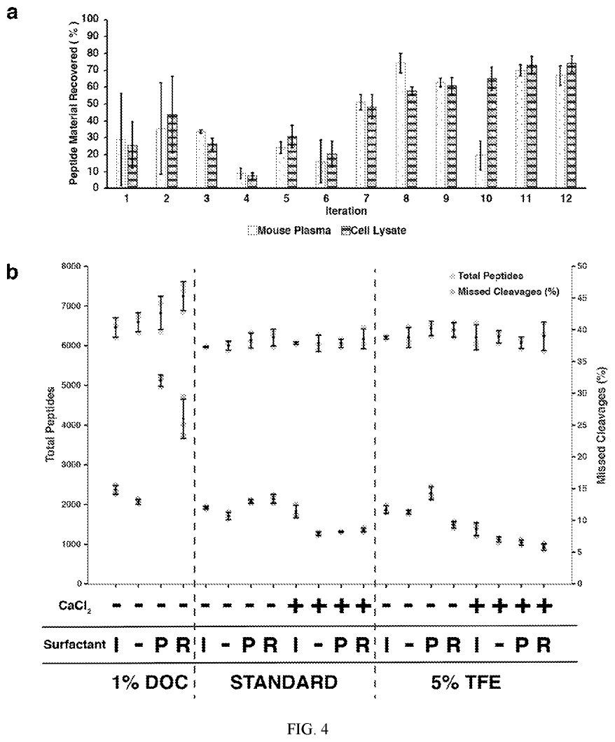

Optimization of digestion efficiency of plasma samples utilizing SP3. For each condition, in triplicate, 100 μg of starting mouse plasma protein was processed starting with lysis through digestion stage with peptide cleanup and followed by analysis on LC-MS2. Various digestion buffer reagent combinations were utilized as indicated in FIG. 4. Sample preparation was performed utilizing the custom 3D printed 96-well scaffold containing 0.2 mL PCR tubes from lysis through digestion, and peptide cleanup was performed as described below (MP3).

Single-pot solid-phase-enhanced sample preparation (SP3) is based on the unbiased immobilization of proteins and peptides on carboxylate modified hydrophilic and hydrophobic coated surfaces mix of beads. Immobilization is promoted through trapping proteins and peptides in an aqueous solvation layer on the bead surface through increasing the concentration of organic additive in solution. Once on the bead surface, proteins and peptides can be processed and rinsed to remove contaminants, such as detergents. Allows for binding of proteins in a non-selective mode. The purified proteins can be eluted from the beads through the addition of an aqueous solution. Resulting peptides can be used in downstream HPLC-based fractionation methods. Or, the peptides can be re-immobilized on the paramagnetic beads for sample clean-up prior to MS-analysis, thus eliminating common sample handling steps.

Multiplexed Single-Pot Solid-Phase enhanced Sample-Preparation (MP3). Lysis, reduction and alkylation were performed as described in methanol/chloroform sample preparation described above. Protein cleanup, digestion, and peptide cleanup sections were performed using SP3 as described previously (McAlister G C, et al. (2014) MultiNotch MS3 Enables Accurate, Sensitive, and Multiplexed Detection of Differential Expression across Cancer Cell Line Proteomes. Anal Chem 86 (14): 7150-7158) except with the following modifications and the addition of the TMT labeling stage. Initial binding of protein to beads was performed at 75% ACN final concentration. Post-binding, two 70% ethanol washes followed by 100% ACN wash were performed. For protein digestion, the protein-bead mixture was resuspended in 90 μL 50 mM HEPES pH 8.5, 10 mM CaCl2. Digestion was performed with Lys-C overnight at room temperature (1:100 protease-to-protein), followed by trypsin (1:25 for trypsin) for 6 h at 37° C. on a thermal mixer. For biological experiments requiring multiple plexes, at this stage a small portion from each sample, or as indicated, was taken to generate a ‘bridge’ sample of 50 μg peptide material for each plex (i.e. 15 samples+bridge per plex). TMT labeling was performed using 50 μg in 80 μL digestion buffer following removal from the magnet immobilized beads. TMT reagent was used at a ratio of 4:1 for cell lysate samples, and 8:1 for mouse & naked mole-rat plasma samples. Excess TMT was quenched with 5% hydroxylamine to a final concentration of 0.3% v/v. Samples per plex were then combined, pipette mixed, and split back out into portions containing no more than 100 μL aqueous volume to bind and cleanup with magnetic beads in 2 mL tubes or 96-well 2 mL plate, to maintain >=95% final ACN concentration to bind peptides to beads. Bead-bound samples were washed 1× with 1000 μL 100% ACN. The portions were eluted 3× with 100 μL 5% ACN, re-combined and dried down in a speedvac overnight. The combined samples were then fractionated offline as described by the HPRP method below. Twelve fractions of set A, resuspended manually in 20 μL MS Loading Buffer and 2 μL per injection, were analyzed on either Orbitrap Lumos Fusion, or Orbitrap Eclipse with FAIMS Pro and RTS, mass spectrometers as indicated.

Automated Multiplexed Protein Profiling Platform (AutoMP3). MP3 method described above was automated using Hamilton Vantage™ liquid handling platform. With the exception of manual lysis step, all of the steps are automated with short hand-on preparation time prior to each step: i) reduction & alkylation, ii) protein cleanup, iii) LysC & Trypsin digestion, iv) bridge generation, v) peptide material normalization prior to TMT labeling, vi) TMT labeling, vii) labeling efficiency check, viii) TMT quenching, ix) Combine-Mix-Split, x) Peptide Cleanup. Eluted cleaned up TMT labeled peptides are then dried down over night and resuspended either for offline HPRP fractionation as described below, or with direct resuspension in MS loading buffer for analysis on either Orbitrap Lumos Fusion, or Orbitrap Eclipse with FAIMS Pro and RTS, mass spectrometers as indicated. Minimal operator intervention was necessary prior to run for each stage to loading the necessary tips, plates and reagents onto the deck.

HPRP Fractionation. The pooled TMT-labeled peptides were fractionated using HPRP. The samples were resuspended in 5% ACN, 10 mM Ammonium Bicarbonate pH 8.0, 95% H2O and fractionated over a ZORBAX extended C18 column (Agilent, 5 μm particles, 4.6 mm inner diameter and 250 mm in length). Peptides were separated on a 75 min linear gradient from 5% to 35% ACN in 10 mM ammonium bicarbonate pH 8.0 at a flow rate of 0.5 mL/min on an Agilent 1260 Infinity pump equipped with a degasser and a diode array detector (set at 214-, 220-, and 254-nm wavelength) from Agilent Technologies (Waldbronn, Germany). The samples were fractionated into a total of 96 fractions and then consolidated in a checkerboard pattern sets of twelve, set A and set B. Samples were dried down under vacuum and reconstituted in MS Loading Buffer for LC-MS/MS processing.

AutoMP3 on Hamilton Vantage™. Liquid classes were optimized, especially for high organic solutions handling. Necessary reagents (lysis buffer, 100 mM DTT, 250 mM IAA) are loaded manually onto respective 96-well plates prior to start. The AutoMP3 protocol begins with 100 μg protein plasma sample with 41.6 μL with a lysis buffer in 200 μL 96-well plates. Followed by the addition of 2 μL 100 mM DTT using the to reduce disulfide bonds and incubated at room temperature for 30 minutes. Then the samples were cysteine alkylated with addition of 2.4 μL 250 mM IAA, incubated in the dark with a plate lid at room temperature for 30 minutes. This was followed up by an additional 2 μL 100 mM DTT to quench the reaction.

While quenching is occurring the deck is prepared for protein binding stage with manual aliquoting of Sera-Mag magnetic beads (GE Healthsciences) freshly resuspended in H2O as well as stock of 100% ACN, 70% EtOH, and digestion buffer in separate 96-well plates (200 μL plate for digestion buffer and 2 mL plate for rest of reagents). The rest of the stage was completed by the liquid handling platform. Stock beads are aliquoted into a custom 3D printed 200 μL plate and supernatant removed using the 96-well Alpaqua magnet. Samples were then thoroughly mixed using the 96-channel head to ensure fast and consistent mixing across the entire plate. Followed by addition of 150 μL 100% ACN to reach final organic concentration of 75% to bind protein to beads, and incubated for 18 minutes, then immobilized for 2 minutes on the Alpaqua magnet. Supernatant was removed while the sample plate is on the magnet, followed by two washes with 100 μL 70% EtOH, and one wash with 100 μL 100% ACN and an additional wash removal step to ensure no ACN is left prior to adding the digestion buffer. The digestion buffer (87 μL 50 mM HEPES, pH 8.5, 10 mM CaCl2) was added once the sample plate was been moved off of the magnet.

This was followed by manual setup for digestion with addition of a 200 μL 96-well plate containing LysC protease. Then, 5 μL of LysC (10 AU resuspended in 5 mL ice-cold water) are added per sample. At each column addition, samples were thoroughly mixed to ensure beads were completely resuspended prior to manually sealing the plate with 8-well strip caps and incubating for 16 hours, 1000 rpm, room temperature using the heater/shaker module. Trypsin was added similarly in the morning at a 1:25 ratio of enzyme to protein (4 μg in 8 μL) and thoroughly mixed to completely resuspend the magnetic beads. And again, manually sealing the plate with 8-well strip caps, followed by an incubation for 6 hours, 1000 rpm, 37° C. using the CPAC module. Peptide BCA was performed automatically on the platform with manual setup of necessary reagents. A worksheet was automatically generated using an in-house python script to direct the 8-channel head to normalize sample volume amounts to be at 50 μg peptide material. If multiple plexes were necessary, at this stage an additional worksheet was generated to create a pooled ‘bridge’ sample of 50 μg peptide material per plex.

In preparation for TMT labeling stage, worksheets were automatically generated to direct the 8-channel head aspiration/dispense steps of the TMTpro reagent as well as combine-mix-split strategy. Necessary reagents were manually set up on the platform including each TMTpro reagent that was aliquoted into a separate 1.7 mL tube and placed in the 32-tube rack. TMT labeling was then started on the platform by first aliquoting portions of TMTpro reagent into a 200 μL 96-well plate to minimize variability of ACN (that the reagent was resuspended in) evaporating during addition to samples. All of the portioned TMTpro reagent is then added at once (24 μL/50 μg plasma sample) to the sample plate using the 96-channel head for rapid, consistent and thorough mixing prior to sealing of the plate using a flexible 96-well mat (Corning) and moved to heater/shaker for 1 hour incubation, room temperature, 300 rpm shaking. Samples are then quenched with 5% hydroxylamine from a reservoir, mixing upon dispensing, followed by 15-minute incubation on the plate rack.

Labeling ratio check evaluations were then be performed by taking 2 μL, using the 8-channel head and a specifically built worksheet, from each sample, then combining, followed by peptide cleanup and analyzing on the instrument. Combine-mix-split strategy is employed: samples, per 16-plex, are combined (with optional input from ratio check results) using the 8-channel head into separate wells in a 2 mL 96-well plate. Each plex is then thoroughly mixed prior to splitting out portions for peptide cleanup.

A new 2 mL plate was prepared with another aliquot of 50 μL of magnetic beads per well in preparation for TMT labelled peptide cleanup, and supernatant is removed using the Alpaqua magnet. Then each plex was split back out into the 2 mL 96-well plate containing the magnetic beads using two columns per plex, which completed the combine-mix-split strategy. Peptides were bound to the beads by reaching >95% organic concentration through addition of 1900 μL 100% ACN, and thorough mixing. Samples were incubated for 18 minutes, followed by incubation on the Alpaqua magnet for 2 minutes to immobilize the beads. A single 1 mL 100% ACN wash was performed. Peptides were then eluted into 1 mL 96-well plate (Eppendorf) three times through addition of 100 μL 5% ACN, thorough mixing and 1000 rpm shaking for 18 minutes on the heater/shaker. For elution, the magnetic beads were then immobilized one more time and eluate is transferred to the 1 mL 96-well plate. For fractionation testing, elution was instead performed using different solutions as indicated in FIG. 11. The split-out portions or fractions of each plex are then recombined, followed by an overnight dry down step using a speedvac. Resulting dried down samples were then either resuspended in 5% ACN/5% formic acid, or fractionated using HPRP, and both were then be analyzed using LC-MS3.

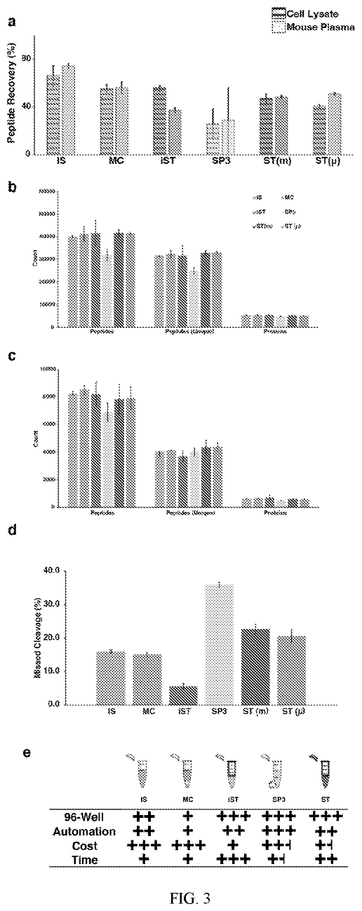

Evaluation of methods compatible for automated proteomics sample preparation. For the first round of comparisons the peptide recovery was evaluated for each of the methods for both plasma and cell lysate (FIG. 3A). IS had the highest recovery, as would be expected since it involved the least sample manipulation and SP3 had the worst recovery. The other methods had comparable performance to MC.

Next, the number of peptide and protein identifications and the missed cleavage rate was evaluated using each sample preparation method (1 μg). The number of proteins identified were similar across all the methods for both cell lysate and mouse plasma sample types, with the exception of SP3, due to the low peptide recovery amount (FIG. 3B-3C). However, the missed cleavage rate was more variable between the different methods (FIG. 3D). The iST method had the lowest mean missed cleavage rate, followed by IS and MC, then ST (u & m) and finally SP3 had the highest.

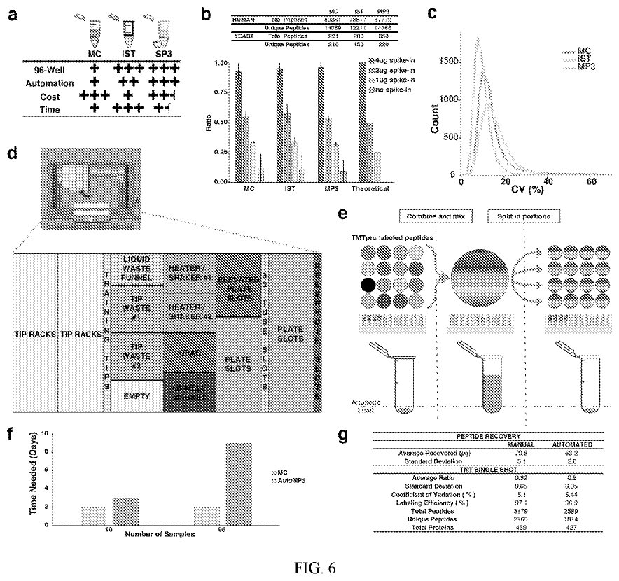

Although SP3 performed the worst in this initial comparison, it was kept in the evaluation for a number of reasons including high score for ease of automation (FIG. 6A, FIG. 3E), published method comparison literature, and the ability optimize the method. While ST had shorter estimated preparation time than SP3, in most regards as a spin filter-based method it did not perform as well as iST, which was faster and has more optimized chemistry and inclusion of processing steps. Therefore, the evaluation of ST (u & m) was discontinued, and focus was directed to evaluating iST and SP3 as top candidates, including MC for reference.

IS was not tested further because it is difficult to automate and limited reagent compatibility due to lack of robust and fast cleanup mechanism. The iST method both has large and optimized method coverage (lysis through TMT labeling steps), and 96-well format, aside from its cost, making it an attractive option. ST did not perform as well as iST and had less method optimization and coverage. No further testing was performed.

Comparison of the methods most amenable to automation (iST vs SP3). Following through some optimization of SP3 steps, much improved peptide recovery and digestion efficiency was achieved (FIG. 4). Based on those results and consideration of automation and feasibility for 96+sample batches preparation the two top candidates were SP3 and iST.

Optimization of SP3: peptide recovery, digestion conditions, TMT labeling. Post SP3 optimization 70 μg recovery for 100 μg starting protein amount, (FIG. 4A) was achieved. This is in-line with previous publications and projections based on the paper comparing protein identifications at microgram cell lysate starting protein amounts comparing SP3, iST, and FASP methods.

The optimizations included varying digestion volume and buffer composition, changing from 2% DMSO to 5% ACN elution solution, varying bead to protein ratios. To address the low digestion efficiency in plasma for SP3 we devised a matrix scheme with 24 conditions to evaluate three fundamental parts of the digestion buffer based on the literature: buffer composition, use of CaCl2, and use of surfactants. For buffer composition, we evaluated 50 mM HEPES pH 8.5, +/−1% Deoxycholate (DOC), +/−5% Trifluoroethanol (TFE). We investigated the effect of addition of 10 mM CaCl2, as well as the addition of Rapigest, PPS, and Invitrosol surfactants.

The presence of small molarity calcium chloride improved digestion efficiency across all conditions (FIG. 4B). While surfactants also improved digestion efficiency, the improvement was minimal when calcium chloride was present. The combination of 1% DOC and 10 mM CaCl2 caused a catastrophic precipitation causing almost complete loss of sample (data not reported). The addition of trifluoroethanol also aided the digestion efficiency. However, for TMT workflows, the use of trifluoroethanol necessitates a cleanup step prior to TMT labeling, since trifluoroethanol is incompatible with the reagent.

The large number of steps involved in a multiplexed TMT experiment makes creating a fully automated method challenging (FIG. 2B). The two most difficult components to automate in a 96-well plate format are the protein cleanup and TMT labeling steps. Methanol chloroform (MC) precipitation is a common approach used for protein cleanup. However, it is not amenable to automation due to difficulties in reproducibly extracting and resuspending the precipitate in a 96-well plate format. The TMT labeling step is challenging to automate for two main reasons. The TMT reagent is easily hydrolyzed in the presence of moisture, and the solubilizing solvent (acetonitrile) is very volatile. Consequently, the reagent needs to be added to the samples with care to minimize evaporation. When scaling up to large numbers of samples, automation of the protein and peptide normalization steps also becomes critical as these steps are time consuming to perform manually.

The parameters considered include performance, estimated ease of adaptability to high throughput multiplexed sample preparation, cost (FIG. 3), as well as processing time for 96 samples (Table 1).

TABLE 1
Time estimates
Rough Time Approximation to Prepare 96 100 μg Plasma Samples Utilizing TMTpro 16-plex/Method

| Time (Hours) | | | | | | | |
|---|---|---|---|---|---|---|---|
| Method Format | IS | MC | ST | iST | SP3 | MP3 | |
| Plexes Processed | TUBES | TUBES | TUBES*\hat{ } | (NHS) | TUBES*\hat{ } | TUBES*\hat{ } | AutoMP3 |
| At A Time | 2 | 2 | 2 | TUBES*\hat{ } | 2 | 2 | PLATE 6 |
| Lysis | 1.0 | 1.0 | 1.0 | 1.0 | 1.0 | 1.0 | 1.0 |
| Protein Quantification | 1.0 | 1.0 | 1.0 | 1.0 | 1.0 | 1.0 | 1.0 |
| Reduce & Alkylate | 1.2 | 1.2 | 1.2 | 0.4 | 1.2 | 1.2 | 1.4 |
| Normalization, Prepare | 1.0 | 2.0 | 2.2 | 1.3 | 1.8 | 1.8 | 0.8 |
| for Digestion/Protein | | | | | | | |
| Cleanup | | | | | | | |
| LysC, Trypsin Digestion | 22.8 | 22.8 | 1.5 | 3.5 | 22.8 | 22.8 | 22.4 |
| Digensted Peptide | 8.0 | 8.0 | 1.0 | N/A | 1.5 | N/A | N/A |
| Cleanup | | | | | | | |
| Dry Down | 16.0 | 16.0 | 16.0 | N/A | 16 | N/A | N/A |
| Peptide Quantification | 1.0 | 1.0 | 1.0 | N/A | 1.0 | 1.0 | 1.0 |
| Peptide Normalization, | 2.7 | 2.7 | 2.7 | 1.7 | 2.7 | 2.7 | 2.2 |
| Bridge Pool, TMT | | | | | | | |
| Labelling | | | | | | | |
| Ratiocheck Prep | 0.8 | 0.8 | 0.8 | 0.3 | 0.8 | 0.8 | 0.7 |
| Ratiocheck Correction | 0.7 | 0.7 | 0.7 | 0.7 | 0.7 | 0.7 | 0.4 |
| Post TMT labelling | 2.0 | 2.0 | 2.0 | 2.0 | 2.0 | 1.5 | 1.7 |
| Cleanup | | | | | | | |
| HPRP Franctionation, | 10.7 | 10.7 | 10.7 | 10.7 | 10.7 | 10.7 | 32.0 |
| Dry Down, Cleanup | | | | | | | |
| Hands-Free Time | 154 | 155 | 91 | 45 | 165 | 113 | 61 |
| Hands-On Time | 53 | 55 | 35 | 23 | 24 | 22 | 3 |
| Total | 207 | 210 | 125 | 67 | 189 | 135 | 65 |

Targets for future improvements
For iST-NHS, 1) digested peptide and TMT labelling are cleaned up in a single stage, and 2) peptide normalization is not part of the workflow.
MP3 and AutoMP3 do not require peptide cleanup immediately after digestion.
*Method can either has 96-well plate format available or can be ported easier than MC or IS. In case of iST automated solution capable of using TMT 11-plex (3 plexes/day not including fractionation), PreON, has become available since 2019.
\hat{ }Method has had published or made publicly available commercially new modifications since evaluation experiments done here.

The two most attractive methods to automate in a 96-well plate format were SP3 and iST (FIG. 6A). Automating the SP3 method was attractive since it can be easily modified, is inherently scalable, and relies on magnetic beads that are very amenable to automation. There has been a growth in usage of magnetic beads for sample preparation in proteomics, including SP3 magnetic bead methods for peptide cleanup using the epMotion 5073m liquid handling platform, label-free studies on an Agilent Bravo, and a combined shotgun proteomics with integrated phosphoproteomics based on SP3 using a Kingfisher Flex. Automating the iST method was also attractive since it had a short start-to-finish time and a low missed cleavage rate (FIG. 3D).

Initial experiments with SP3 resulted in both poor recovery and digestion efficiency (FIG. 3). After many iterations to optimize workflow methodology (Table 2) peptide recovery was increased from 25% to 70% (FIG. 4A).

TABLE 2
Summary of optimization of workflow methodology.

| | Methodology |
|---|---|
| Iteration#1 | Perform the standard methods as described in Hughes et al, Moll Syst Biol, |
| | 10, 757, 2014 |
| Iteration#2 | No pellet disruption during washes. Issue - digestion buffer dried out during |
| | digestion, eluted with 50 μL 2% DMSO twice |
| Iteration#3 | Increased digestion volume to 200 μL 50 mM HEPES pH 8.5 |
| Iteration#4 | Decreased digestion volume to 100 μL, switched to lysis buffer 3% SDS, 50 |
| | mM HEPES pH 8.5, 75 mM NaCl, halved bead amount, halved starting |
| | sample volume to 100 μL, decreased wash volumes to 200 μL |
| Iteration #5 | Decreased digestion volume to 10 μL, switched to PCR tubes for |
| | preparation, decreased starting sample volume to 51 μL, eluted 1x with 50 |
| | μL 2% DMSO, 1x with 100 μL 2% DMSO |
| Iteration#6 | Used elution volumes of 50 μL, and 100 μL 2% DMSO (total elution |
| | volume 150 μL), manually pipetted 30 times at start of elution incubation |
| Iteration#7 | Increased bead content 3x, performed washes while sample-bead mixture |
| | was magnet immobilized, and eluted with sonication |
| Iteration#8 | Set bead:protein ratio of 10:1 w:w, eluted using 2x 100 μL 2% DMSO |
| | using 5 minute sonication, vortexing and benchtop incubation |
| Iteration#9 - | Repeated Iteration#8 |
| Iteration 10# | LysC digested overnight, followed by trypsin for 6 hours, plasma samples |
| | had slightly acidic pH during peptide cleanup |
| Iteration#11 | Increased digestion volume to 100 μL and buffer composition to 50 mM |
| | HEPES pH 8.5, skipped peptide cleanup |
| Iteration#12 | Buffer composition changed to 50 mM Ammonium Bicarbonate, utilized 2 |
| | μL LysC (1:100, protease:protein), 8 μL trypsin (1:25, protease:protein), |
| | pipetting digest off beads via magnet immobilization and rinsed with |
| | another elution of 100 μL 50 mM ammonium bicarbonate |

To optimize the digestion efficiency, a matrix of 24 different conditions was prepared, investigating the effect of adding 1% Deoxycholate (DOC), +/−5% Trifluoroethanol (TFE), Rapigest, PPS, and Invitrosol surfactants as well as CaCl2. The greatest improvement was observed by the addition of CaCl2 and 5% TFE. With these changes the missed cleavage rate decreased from 30% to <5% (FIG. 4B). Unexpectedly, in absence of CaCl2, 1% DOC with Rapigest or PPS did not improve digestion efficiency with the former and made it worse with the latter.

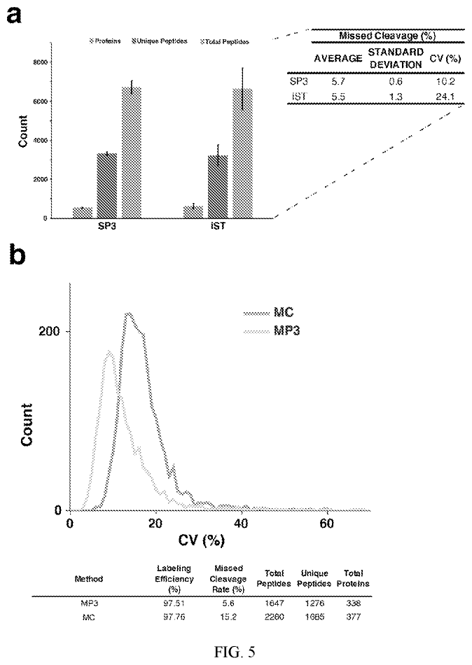

To decide whether to automate SP3 or iST, the performance of these two methods were compared using both label-free and TMT workflows. In the label-free experiment, SP3 and iST methods had similar performance for mouse plasma samples, both in terms of the average number of peptide and protein identifications as well as missed cleavage rates. However, there was higher variability in iST method (FIG. 5A). Labeling efficiency of MC, iST, and multiplexed SP3 (MP3) was evaluated on human cell lysates. Evaluation of labeling efficiency (%) MC vs iST vs MP3, yeast spike-in experiment setup, quantified total peptides, unique peptides, and total proteins (Tables 3-5). The methods performed similarly, although iST performed slightly worse (Table 3). Accuracy and precision, based on the yeast spike-in peptides were similar between MC, iST, and MP3 (FIG. 6B, Table 5).

TABLE 3
TMT labeling efficiency (%) for MP3, iST, and
MC on human cell lysate samples (n = 1).

| | Total | Unique | Total | Labeling |
|---|---|---|---|---|
| Method | Peptides | Peptides | Proteins | Efficiency (%) |
| MC | 21087 | 18204 | 3754 | 99.4 |
| iST | 18757 | 15885 | 3367 | 98 |
| MP3 | 21366 | 18330 | 3868 | 99.1 |

A TMT-based two organism (human/yeast) spike-in experiment was performed (Table 3) to compare the precision and accuracy of the MP3 and iST against MC.

TABLE 4
Yeast spike-in experimental design to evaluate accuracy and precision across the three methods.

| Sample | S1 | S2 | S3 | S4 | S5 | S6 | S7 | S8 | S9 | S10 | S11 |
|---|---|---|---|---|---|---|---|---|---|---|---|
| human cell lysate (μg) | 100 | 100 | 100 | 100 | 100 | 100 | 100 | 100 | 100 | 100 | 100 |
| yeast spike-in (μg) | 4 | 2 | 1 | 0 | 4 | 2 | 1 | 0 | 4 | 2 | 1 |

TABLE 5
Both yeast-only and human-only quantified total peptides, unique
peptides, and total proteins per method evaluated (n = 1).

| | | | |
|---|---|---|---|
| Method | Total Peptides | Unique Peptides | Total Proteins |

Quantified Yeast Peptides & Proteins

| | | | |
|---|---|---|---|
| MC | 291 | 218 | 82 |
| iST | 200 | 153 | 68 |
| MP3 | 353 | 220 | 90 |

Quantified Human Peptides & Proteins

| | | | |
|---|---|---|---|
| MC | 89361 | 14089 | 2740 |
| iST | 78817 | 12211 | 2497 |
| MP3 | 87772 | 14068 | 2781 |

It was found that MP3 had the smallest coefficient of variation when considering human only peptides (FIG. 6C). It was ultimately chosen to automate MP3, rather than iST, since a lower coefficient of variation was observed (FIG. 6C) and because the quantity of beads could be scaled to different amounts of sample very easily. A head-to-head comparison was performed to determine whether MP3 had similar performance to our standard MC precipitation in the desired sample matrix (mouse plasma). While the proteins quantified differed within an acceptable range between the two techniques, MP3 had a much lower missed cleavage rate and lower variation (FIG. 5B). Therefore, automating MP3 was determined to be more favorable.

### Example 2: Automation of Multiplexed Proteome Profiling Platform (AutoMP3)

The MP3 method was automated end-to-end on a Hamilton Vantage™ robot. There were a number of requirements for the automated workflow. One, sufficient capacity for consumables such that end-to-end automation of each of the steps (FIG. 2B) was possible. Two, the ability to accurately and precisely pipette different liquid types in volumes from 1 μL to 1 mL for the entirety of a 96-well plate and on a per-well basis. Three, the ability to accommodate and control other devices in the workflow (magnetic rack, heaters, shakers). Four, the ability to move labware and lids on and off the devices. Finally, robust hardware and software that could operate for long periods of time without end-user intervention was needed. With these requirements in mind, the Hamilton Vantage™ for the liquid handling robot was selected.

For the Vantage™ configuration, two heads were selected: a 96-well fixed-head capable of picking up volumes between 1-1,000 μL, and an individually operated multichannel head consisting of eight 1-1,000 μL channels. A two-meter deck-length option was selected for its large capacity. The internal plate gripper (IPG), which is capable of transferring items around most of the deck space, was a crucial element of the platform. Additionally, two heater-shakers, a cold plate air cooled (CPAC) shaker, and a magnetic rack for magnetic bead immobilization were added. FIG. 6D shows the deck layout. The Vantage™ platform included software, Instinct V, that allows granular control over aspiration, dispensing, and mixing steps as well as customizable per well sample handling worksheets.

The large deck was capable of fitting all of the tips, plates, liquid and tip waste compartments, and modular devices necessary for thre method. The deck is spacious enough for future additions. Aside from bulk processing of 96 samples at a time, the presence of an 8-channel head allowed for the freedom to finely control key stages of the sample processing workflow. First, various configurations of TMTpro, or alternate reagents could be patterned across the 96-well plate. Second, it was possible to normalize sample content at the protein, peptide, and TMT-labeling-stages. Third, it was possible to generate bridge samples per plex at any stage.

All of the liquid handling steps were tested and optimized, especially for small volume handling steps such as the addition of LysC and trypsin for the digestion. The liquids used in the protocol vary considerably in physical properties, from the viscus bead slurry to the volatile 100% acetonitrile. To meet this challenge the Vantage™ pipetting channels and software allow for detailed control of all the parameters of the pipetting process. Thorough mixing is critical for a number of the protocol steps, in particular the resuspension of the magnetic beads with protein lysate and the addition of the TMT reagent for peptide labeling. Care was taken to mix reagents as quickly as possible, but without causing bubbles to form or residual liquid to be retained in the tips.

The main challenges with automating MP3 were variability, TMT labelling and cleanup post TMT. The sequence timing for the pipetting steps and plate movements were carefully considered to keep well variation low. The TMT labeling stage was difficult due to the high vapor pressure of acetonitrile which the TMT reagents were dissolved in. To address this, liquid classes for the steps involving acetonitrile were optimized. Cleanup post TMT labelling was a major challenge since the total volume for a 16-plex experiment results in very large volumes (for example, 1.6 mL aqueous volume requires 30.4 mL 100% acetonitrile for the peptide binding stage). Large volumes are difficult to deal with in a 96-well plate format. To solve this problem, a combine-mix-split strategy was devised to keep the volumes low (FIG. 6E). This strategy involved first combining and mixing all the TMT labelled samples in a plex and then splitting them into multiple wells for smaller volume cleanup before being combined again after cleanup.

In some embodiments, the method further comprises mixing the labelled peptides or polypeptides. In some embodiments, the mixing is by aspiration and dispensing. In some embodiments, the mixing is by vibration. In some embodiments, the mixing is by passive diffusion.

In some embodiments, one plex is split into two or more portions. In some embodiments, the plex-portions are substantially homologous. In some embodiments, each plex-portion is substantially homogeneous. In some embodiment, one plex is split into a plurality of plex-portions. In some embodiments, the plurality of plex-portions have substantially equal volumes. In some embodiments, the plurality of plex-poions have identical volumes. In some embodiments, the plurality of plex-portions have unequal volumes.

In some embodiments, increasing the number of samples increases the number of plex-portions due to an increased volume of samples. For examples, 16 samples may be split into about 16 plex-portions and the volume of each portion is about 91 μL.

In some embodiments, the volume of each plex-portion is less than about 1 mL. In some embodiments, the volume of each portion is about 1 μL to about 100 μL. In some embodiments, the volume of each portion is more than 6 μL. In some embodiments, the volume of each portion is less than 100 μL. In some embodiments, the volume of each portion is about 5 μL. In some embodiments, the volume of each portion is about 10 μL. In some embodiments, the volume of each portion is about 20 μL. In some embodiments, the volume of each portion is about 30 μL. In some embodiments, the volume of each portion is about 40 μL. In some embodiments, the volume of each portion is about 50 μL. In some embodiments, the volume of each portion is about 60 μL. In some embodiments, the volume of each portion is about 70 μL. In some embodiments, the volume of each portion is about 80 μL. In some embodiments, the volume of each portion is about 90 μL. In some embodiments, the volume of each portion is about 100 μL.

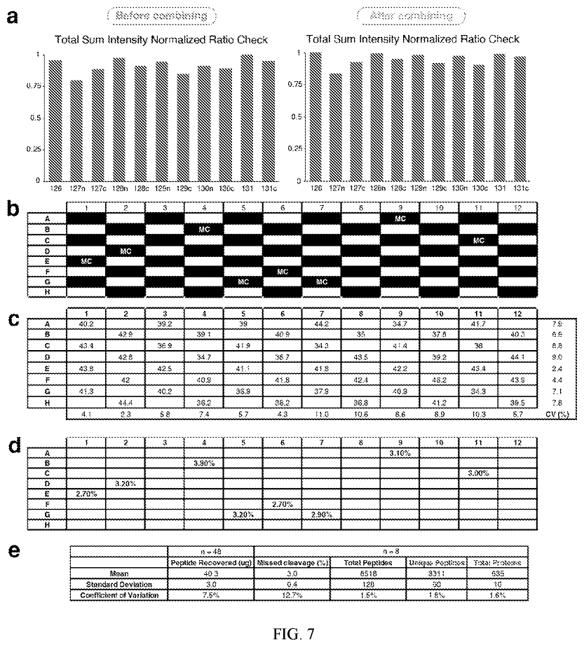

To validate the combine-mix-split strategy for TMT peptide cleanup, manual and automated preparations of mouse plasma labelled with TMTpro were compared using 16 technical replicates all mixed at 1:1 ratios. Comparable low variability was observed between the standard and combine-mix-split approaches (FIG. 7A). The combine-mix-split approach required more pipetting steps but is easily performed on the automated liquid handler and avoids variable recovery that is inevitable with individually cleaned up samples. Cleanup post TMT is a notoriously difficult step to automate due to the large quantities of material involved. Other workflows perform peptide-level cleanup on each individual sample. While this avoids the problem of large sample volumes, it leads to more variation. Here, it has been demonstrated that this combine-mix-split approach is a solution for automation of peptide cleanup post-TMT labeling.

The Hamilton Vantage™ liquid-handler system performed all of the liquid handling for the sample preparation, with limited interactions from the end user. Plasma samples were aliquoted into plates, then reagents for reduction and alkylation are added and mixed automatically. Incubation steps were timed and controlled by the system. The protein purification steps were accomplished utilizing Sera-Mag magnetic beads. Throughout purification, the Hamilton Vantage™ was able to move the sample plate on and off of the magnetic rack to immobilize the beads, as well as to perform all of the washing and elution steps. The proteins were digested with LysC and trypsin, and the peptide concentration is measured via a bicinchoninic acid (BCA) assay (Thermo Fisher Scientific). The system used the individual concentration values to normalize each sample so that a consistent mass of 50 μg of peptides were labeled. The TMTpro labeling was automated, with the ability to adjust for the number of samples and degree of sample multiplexing. The post-labeling cleanup and combine-mix-split strategy took advantage of the Vantage™'s ability to handle deep-well plates and pipette larger volumes of 100% acetonitrile. TMT labeling, and cleanup were followed by a dry down step using a speedvac overnight. Plexes can then either be fractionated or resuspended in 5% acetonitrile/5% formic acid and directly analyzed on the mass spectrometer. A detailed description of all the steps is included herein.

The robustness of the automated MP3 method was evaluated on the Vantage™ across an entire 96-well plate. Samples prepared in a 96-well plate using a checkerboard pattern (FIG. 7B) resulted in low variation in overall peptide recovery (CV=7.5%) and missed cleavage rates (3.0%) (FIG. 7B-E). Variation appeared to be random, with no systematic variation on a row-by-row or column-by-column basis. Further optimization for peptide recovery throughout the sample preparation steps was performed (mixing, aliquot and dispensing speeds, and pipette tip depth position). It was found that switching to 50 mM HEPES pH 8.5, 10 mM CaCl2 for the digestion buffer allowed for skipping the peptide cleanup prior to TMT labeling. This increased the average peptide recovery and lowered variability (FIG. 6G).

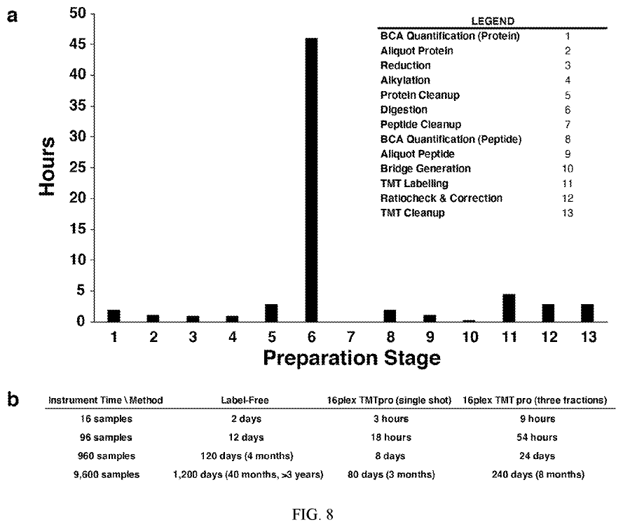

Transferring the MP3 method to an automated liquid handling platform resulted in significant time savings for sample preparation (FIG. 8A, Table 1). Compared to MC, AutoMP3 processes 96 samples approximately four times faster (FIG. 6F). Using TMTpro multiplexing (16-plex) instead of a label-free approach allows the mass spectrometer to keep pace with this increase in sample preparation throughput. For 96 samples, using TMTpro requires less than a day of instrument time, versus 12 days for label-free analysis (FIG. 8B).

### Example 3: Maximizing Single Shot TMT Plasma Proteome Coverage Using Orbitrap Eclipse Coupled to a FAIMS Pro with Real Time Search MS2 Filter

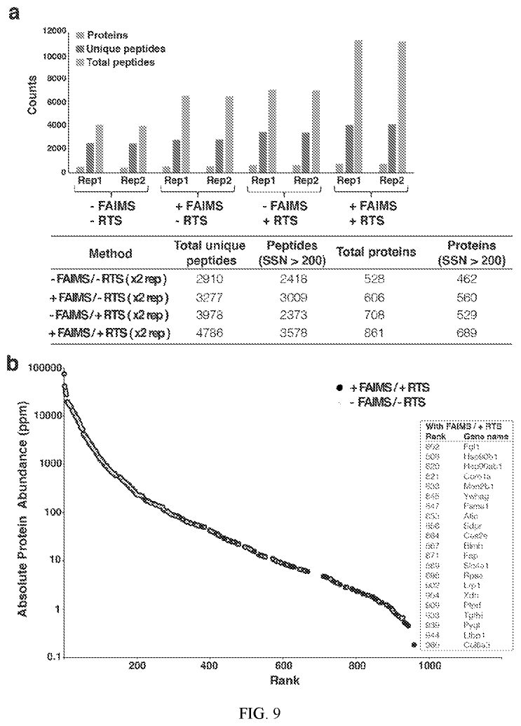

In order to maximize throughput, performing single shot analysis was optimized while still maximizing the number of protein identifications (IDs) from blood plasma. To that end, the effect of including Real Time Search (RTS) and high-field asymmetric waveform ion mobility (FAIMS Pro) was evaluated. Previously both of these MS tools have been shown to increase the number of peptide and protein identifications, but neither of those studies tested the performance of these tools in the context of blood plasma. Extensive optimization was needed of both ion times and AGC targets for the MS3 scan in combination with testing out different compensation voltage (CV) settings (Table 6). The greatest number of quantified plasma peptides was achieved using CVs of −40, −50, −60, and −70. A pre-release version of the instrument control software was utilized for the Orbitrap Eclipse that allowed RTS to be used across multiple CVs in the same run (Tune 3.4). The addition of FAIMS Pro and RTS increased the number of peptide IDs over 64% and quantified peptides 48%, while the number of proteins IDs increased 63% and quantified proteins 49% (FIG. 9A, Table 6).

TABLE 6
Evaluation of FAIMS Pro Compensation Voltages (CV), injection times and real time search parameters on the
Eclipse. Analyzed using 25 cm Aurora IonOpticks column, 1.28 μg mouse plasma TMT-16plex per injection.

| | | | | MS 3 | RTS | | | Unique pep. | Unique pep. | Unique prot. | Total Peptides |
|---|---|---|---|---|---|---|---|---|---|---|---|
| Instrument method with | MS2 | MS2 | MAS3 | Inj. Time | Xcorr/ | # of | # of | (1% pep. | (1% prot. | (1% prot. | with signal to |
| (FAIMS CVs)/inclusion list | AGC | IT | AGC | (ms) | dCN | MS2 | MS3 | FDR) | FDR) | FDR) | noise (>200) |
| [symbol] ( [symbol] , [symbol] , [symbol] )/ [symbol] | [symbol] | [symbol] | [symbol] | [symbol] | [symbol] | [symbol] | [symbol] | [symbol] | [symbol] | [symbol] | [symbol] |
| [symbol] ( [symbol] , [symbol] , [symbol] )/ [symbol] | [symbol] | [symbol] | [symbol] | [symbol] | [symbol] | [symbol] | [symbol] | [symbol] | [symbol] | [symbol] | [symbol] |
| [symbol] ( [symbol] , [symbol] , [symbol] )/ [symbol] | [symbol] | [symbol] | [symbol] | [symbol] | [symbol] | [symbol] | [symbol] | [symbol] | [symbol] | [symbol] | [symbol] |
| [symbol] ( [symbol] , [symbol] , [symbol] )/ [symbol] | [symbol] | [symbol] | [symbol] | [symbol] | [symbol] | [symbol] | [symbol] | [symbol] | [symbol] | [symbol] | [symbol] |
| [symbol] ( [symbol] , [symbol] , [symbol] )/ [symbol] | [symbol] | [symbol] | [symbol] | [symbol] | [symbol] | [symbol] | [symbol] | [symbol] | [symbol] | [symbol] | [symbol] |
| [symbol] ( [symbol] , [symbol] , [symbol] )/ [symbol] | [symbol] | [symbol] | [symbol] | [symbol] | [symbol] | [symbol] | [symbol] | [symbol] | [symbol] | [symbol] | [symbol] |
| [symbol] ( [symbol] , [symbol] , [symbol] )/ [symbol] | [symbol] | [symbol] | [symbol] | [symbol] | [symbol] | [symbol] | [symbol] | [symbol] | [symbol] | [symbol] | [symbol] |
| [symbol] ( [symbol] , [symbol] , [symbol] )/ [symbol] | [symbol] | [symbol] | [symbol] | [symbol] | [symbol] | [symbol] | [symbol] | [symbol] | [symbol] | [symbol] | [symbol] |
| [symbol] ( [symbol] , [symbol] , [symbol] )/ [symbol] | [symbol] | [symbol] | [symbol] | [symbol] | [symbol] | [symbol] | [symbol] | [symbol] | [symbol] | [symbol] | [symbol] |
| [symbol] ( [symbol] , [symbol] , [symbol] )/ [symbol] | [symbol] | [symbol] | [symbol] | [symbol] | [symbol] | [symbol] | [symbol] | [symbol] | [symbol] | [symbol] | [symbol] |
| [symbol] ( [symbol] , [symbol] , [symbol] )/ [symbol] | [symbol] | [symbol] | [symbol] | [symbol] | [symbol] | [symbol] | [symbol] | [symbol] | [symbol] | [symbol] | [symbol] |
| [symbol] ( [symbol] , [symbol] , [symbol] )/ [symbol] | [symbol] | [symbol] | [symbol] | [symbol] | [symbol] | [symbol] | [symbol] | [symbol] | [symbol] | [symbol] | [symbol] |

[symbol]

indicates data missing or illegible when filed

The addition of FAIMS Pro alone resulted in a 13% increase in peptide IDs, while the addition of RTS alone resulted in a 37% increase (FIG. 9A). Furthermore, the combination of FAIMS Pro and RTS enabled quantitative measurements of lower abundant analytes (FIG. 9B) such as Prolow-density lipoprotein receptor-related protein 1 (Lrp1), Glycogen phosphorylase (Pygl), and Collagen, type VI, alpha 3 (Col63a) that were not identified without the FAIMS Pro-RTS combo. The concentration of Lrp1 has been reported to be present in 4.7-5 pmol/ml in C57BL/6/CRL and C57BL/6J when measured with gold standard MRM assays, while in the same study analytes such as Pygl were reported as not detected in several of their tested mouse strains.

### Evaluation of the Use of an Inclusion List During LC-MS Acquisition

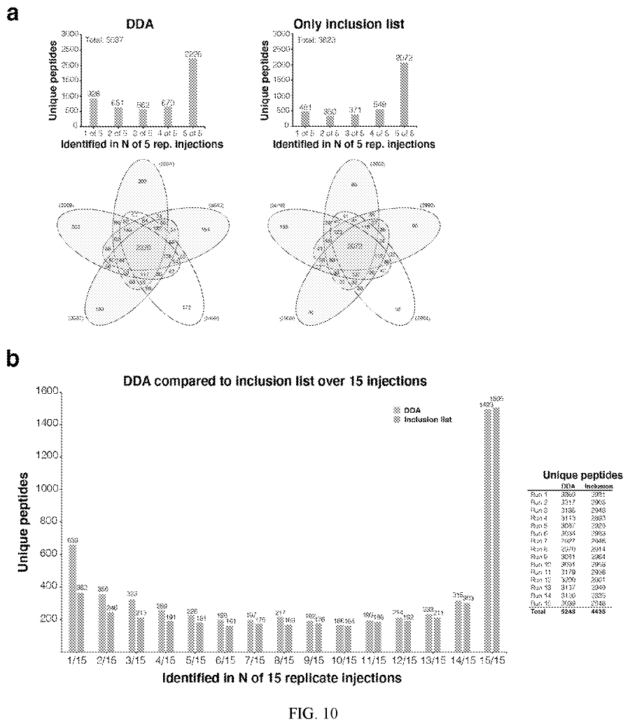

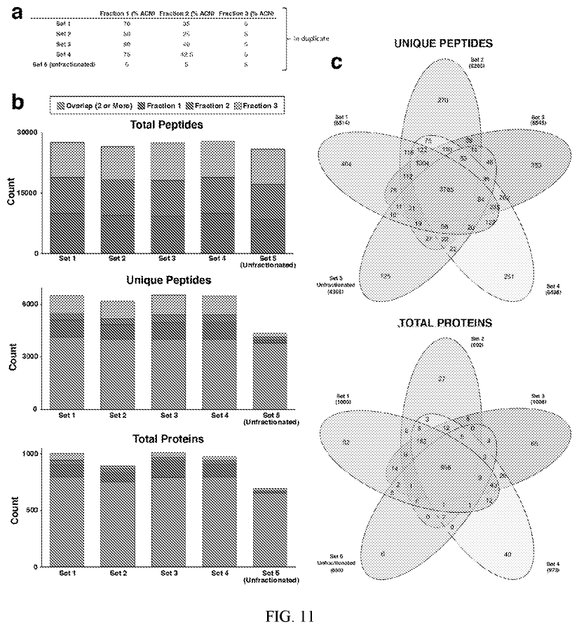

One of the main issues when combining data from multiple TMT-plexes is that the overlap in identified and quantified peptides decreases as the number of plexes increases. While data dependent acquisition (DDA) is the most efficient way to quickly produce a large number of peptide IDs, it often lacks adequate peptide identification reproducibility due to the way it stochastically samples precursors. This becomes even more challenging in a sample matrix like plasma due to the large dynamic range of plasma proteins. In order to maximize chances of comparing protein and peptide abundances based on the measurement of the same peptides across multiple TMT-plexes, the potential effect of the usage of inclusion lists was evaluated across series of both a smaller (n=5) and larger (n=15) set of replicate injections. For both of the test series, the target masses on the inclusion list were derived by selecting the best scoring peptides from five initial DDA runs of the sample series. For the smaller sample set, the largest coverage and greatest overlap was achieved using a regular DDA method without an inclusion list: consistent measurement of 2,226 peptides (amounting to 44% overlap out of total unique peptides identified) across five single plasma injections (FIG. 10A). The DDA method with the inclusion list only covered 2,072 peptides (54% overlap), corresponding to detecting 38% of the actual targeted masses. However, when analyzing the larger sample set consisting of fifteen 16-plexes, the inclusion list approach outperformed the DDA and resulted in measurement of 1,505 peptides (34% overlap) compared to 1,493 peptides for DDA across the fifteen injections (FIG. 10B). The inclusion list approach for fifteen injections corresponded to a coverage of 31% of the targeted masses. Evaluation of additional MS2 and MS3 parameters, such as mass window accuracy, injection times, and AGC settings, when using an inclusion list, could potentially further improve the overlap, but these parameters were beyond the scope of this study. Improved peptide coverage across all runs mitigates many of the complexities caused by missing values in mass spectrometry proteomics experiments. Although these problems are more severe in label-free than multiplexed experiments, the problem does persist when combining multiple plexes.

### Evaluation of the Benefit of Fractionation

Fractionation yields additional proteome depth for complex samples such as plasma. Fractionation was easily implemented with the AutoMP3 platform using a modified version of the peptide cleanup protocol, where instead of eluting peptides with a single buffer, three (or more) elution buffers could be applied (for example, a gradient of high to low concentration of acetonitrile) and each transferred to separate 96-well plates. Fractionating using AutoMP3 in the 96-well format allows for higher throughput than our standard high-pressure reverse phase (HPRP) fractionation setup. As with other steps, fractionation was designed in a modular fashion. The effect of fractionating label-free mouse plasma samples into three parts was evaluated, using various acetonitrile-based elution gradients. Fractionated sets were compared against three single shot injections of an unfractionated sample (FIG. 11A). While within the various fractionation gradients we did not see a major difference of unique and total proteins identified, the difference between fractionated and unfractionated sets was noticeable. An increase of 29.5% and 26.7% more unique peptides and total proteins identified was observed, respectively (FIG. 11B).

### Evaluation of Different LC-MS Column Set-Ups

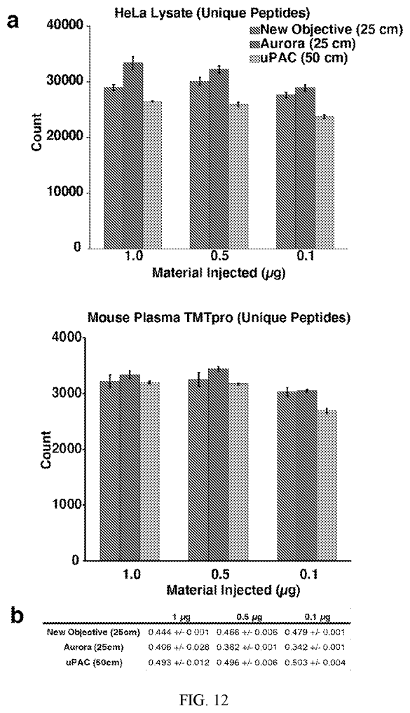

When considering the analysis of thousands of samples, it is critical to have robust chromatography. This is of particular importance when using an inclusion list method with retention time scheduling. Three LC columns from different suppliers were evaluated: New Objective (NO, 100 μm ID, C18 1.7 μm 130 Å, 25 cm bed), IonOpticks Aurora (IO, 75 μm ID, C18 1.6 μm 120 Å, 25 cm bed), and Pharmafluidics uPAC (uPAC, 40 μm ID, C18 pillar surface, 50 cm bed) (FIG. 12). Pressure on the system was highest for Aurora because that column has the smallest packed C18 resin size (1.6 Å). The Aurora column was followed by the New Objective, and uPAC chip columns, the latter having approximately 50%-80% reduction in pressure (bar). Unique peptide identification rates show a clear benefit with the Aurora column (FIG. 12A) for both label-free HeLa cell lysates and TMTpro mouse plasma single shot injections. The median peak width was the narrowest with the Aurora column (0.41 min) compared to broader peaks for New Objective, and uPAC, (0.44 min, and 0.49 min respectively) for 1 μg injection TMTpro labelled mouse plasma (FIG. 12B). It is worth noting that the median peak width increased as the amount of material injected decreased for New Objective, while the reverse occurred with Aurora column. The uPAC chip maintained relatively constant peak width for each of the peptide amounts injected.

Each column has its advantages and disadvantages (lifetime, pressure during runtime, capacity, and resolution) and each requires different optimizations. Initial comparison shows that, on average, the IonOpticks Aurora 25 cm column performed the best in our hands for both label-free and TMTpro labelled samples (FIG. 12).

### Example 4: Analysis of Circadian Rhythm Naked Mole-Rat Samples Using AutoMP3

Sample Collection for Naked Mole-rat Circadian Rhythm Study. Naked mole-rats were raised under Association for Assessment and Accreditation of Laboratory Animal Care International [AAALAC] standards. Animal protocol A10199 was approved by the Buck Institute For Research on Aging Institutional Animal Care and Use Committee. Two-year-old, one male and one female, was sacrificed using isoflurane inhalation followed by cardiac exsanguination every 2 hours over a 48 hr period. Whole blood was collected using EDTA as an anticoagulant and samples were centrifuged at 5000×rpm for 5 minutes. Plasma was separated from whole blood, aliquoted, flash frozen and stored at −80° C. until use. In total 48 naked mole-rats were euthanized in this study. Corresponding male and female timepoints were treated as duplicates in the analysis.

Sample Collection for UV Treated Mice and Naked mole-rats. Naked mole-rats and Skh1 hairless mice were raised under Association for Assessment and Accreditation of Laboratory Animal Care International [AAALAC] standards. Animal protocol A10139 was approved by the Buck Institute For Research on Aging Institutional Animal Care and Use Committee. Naked mole-rats were provided ad libitum access to food and mice were provided ad libitum access to food and water. Physiologically age-matched male naked mole-rats and Skh1 hairless mice were irradiated with the same dose of UV light (180 mJ/cm2). Two-year-old male naked mole-rats and 2-month-old male mice were placed on a rotating platform under 8 UV lamps emitting 72.6% UVB and 27.4% UVA. UV emission was measured using an ultraviolet sensor. Control animals were sham treated on the UV exposure platform. Animals were sacrificed via isoflurane and cardiac exsanguination 48-(2 days) and 168-hours (1 week) after UV exposure. Whole blood was collected using EDTA as an anticoagulant and samples were centrifuged at 5000×rpm for 5 minutes. Plasma was separated from whole blood, aliquoted, flash frozen and stored at −80° C. until use.

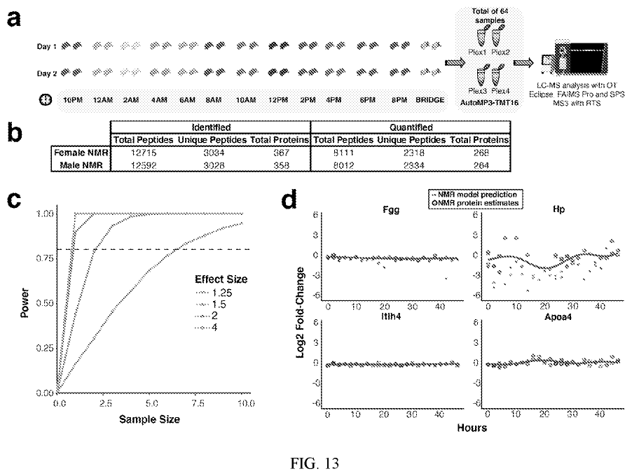

Molecular circadian rhythm changes were investigated in naked mole-rats using plasma samples, collected every 2 hours over a 48 hr period from young male and female naked mole-rats (FIG. 13A). Large changes had previously been reported in skin. The 64 samples were prepared with the AutoMP3 workflow, which took less than two days with just two hours of hands-on time followed by 16 hours of instrument time to analyze. It was found that 367 and 358 proteins in female and male naked mole-rats were identified, respectively. Additionally, 268 proteins in females and 264 in males were quantified (FIG. 13B).

A power analysis was performed to determine the minimum number of replicates needed to detect circadian rhythm changes at several magnitudes over a 48-hour period (FIG. 13C). The power analysis suggested that 48 samples, in this design, should be sufficient with >80% power to detect sinusoidal patterns with relatively large changes throughout the day (Fold-changes>2). However, <1% of proteins were observed that fit the circadian rhythm pattern at an FDR corrected p-value cutoff of 0.01. Several selected proteins as an example are shown in FIG. 13D.

### Example 5: Analysis of Mice and Naked Mole-Rats Treated with UV Irradiation Using AutoMP3

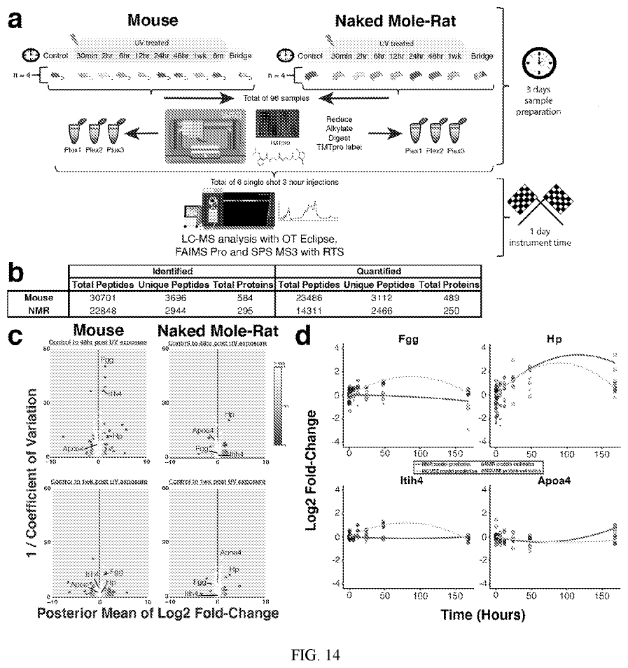

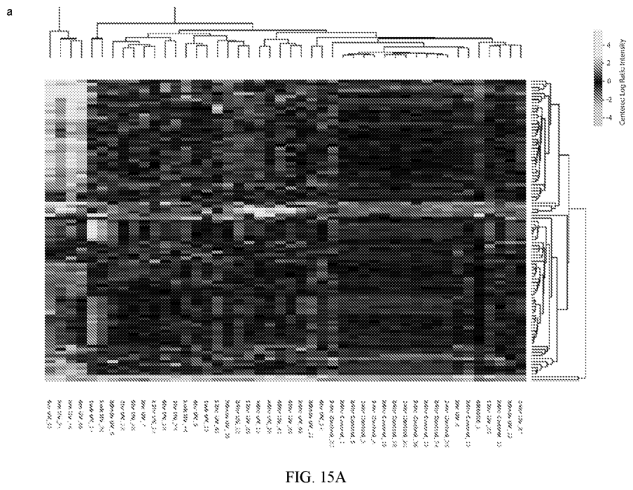

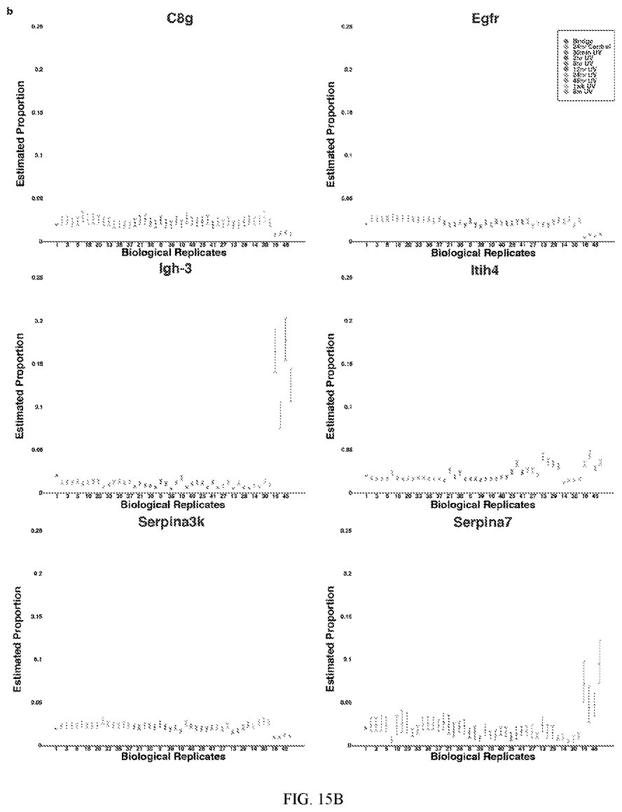

Two-month-old, male Skh1 hairless mice and two-year-old, male naked mole-rats were treated with UV radiation (180 mJ/cm2) and sacrificed at both 48 hours and 7 days after a single UV exposure (FIG. 14A). As part of the study, samples from mice treated with UV exposure 2×/week for a 6-month period were also analyzed. Resulting protein changes post 6-month UV treatment were significantly larger than that of the rest of the time points (FIG. 15). However, those changes are difficult to interpret without 6-month control untreated samples to factor out aging effects. In the end only short-term changes were considered in the final analysis (Control, 30 minutes to 1-week post UV exposure). Blood was collected by cardiac exsanguination and the plasma was separated for analysis. Sample preparation using AutoMP3 of 96 samples took three days with just 2.5 hours of hands-on time, followed by 21 hours of instrument time. From this study, 584 mouse and 295 naked mole-rat plasma proteins were identified, respectively. (FIG. 14B). Although relatively few plasma proteins showed significant changes, the global plasma proteome pattern produced after UV exposure was quite distinct between mice and naked mole-rats. In mice, a quadratic temporal relationship was clearly evident and UV exposure induced rapid changes in responsive proteins with the greatest amplitude of change observed 48 hours post exposure. By seven days, most of these protein levels had returned back to baseline levels (Tables 7). In contrast to the observed time course of the mouse plasma proteome profile, naked mole-rats generally showed a markedly attenuated response, with more modest fluctuations and, unlike mice, did not show response resolution even after 1 week.

TABLE 7
Protein changes detected, observed patterns from literature for mouse and naked mole-rat, and function respectively.

| | | | | ACUTE PHASE | |
|---|---|---|---|---|---|
| PROTEIN | GENE | MOUSE | NAKED MOLE-RAT | PATHWAY | FUNCTION |
| Serum amyloid A | Saa | Significant increase at 4 [symbol] | Not Detected | + | i) Chemotactic recruitment |
| | | hours post UV exposure | | | of immune cells to sites of |
| | | SAA1, SAA2, and SAA4 | | | injury |
| | | | | | ii) Induction of enzymes |
| | | | | | that degrade extracellular |
| | | | | | matrix |
| | | | | | iii) Downregulation of |
| | | | | | inflammatory processes |
| | | | | | iv) Lipid metabolism and |
| | | | | | transport |
| | | | | | v) Binding and scavenging |
| | | | | | of cholesterol |
| Haptogoblin | Hp | Significant increase at 4 [symbol] | Significant increase at 48 | + | i) Involved in defenses |
| | | hours post UV exposure | hours post UV exposure, | | against inflammation |
| | | Decrease at 7 days post | and remains at that level | | ii) Natural antagonist for |
| | | UV exposure | at 7 days post UV | | receptor-ligand activation |
| | | | exposure | | of the immune system system |
| | | | | | iii) Increase anti- |
| | | | | | inflammatory [symbol] |
| | | | | | iv) Binding of free |
| | | | | | hemoglobin |
| | | | | | v) Stabilizing iron |
| | | | | | vi) Extracellular chaperone |
| | | | | | vii) Antioxidant |
| | | | | | viii) Inhibition of |
| | | | | | granulocyte chemotaxis |
| | | | | | and phagocytosis |
| C-reactive protein | Crp | Peak at 48 hours post UV | Still rising at 7 days post | + | i) Promotes binding of |
| | | exposure | UV exposure | | complement |
| | | | | | ii) Induction of cytokines |
| | | | | | iii) Induction of chemotaxis |
| | | | | | iv) Modulation of |
| | | | | | neutrophils |
| Complement [symbol] | C3, C [symbol] | C3 significant peak at 48 | C [symbol] significant decline at | + | |
| | | hours post UV exposure | 4 [symbol] hours post UV | | |
| | | and declines to baseline at | exposure and remains at | | |
| | | 7 days post UV exposure. | low level. | | |
| | | C [symbol] unchanged 0-7 days | C [symbol] significant peak at 4 [symbol] | | |
| | | with UV exposure | hours post UV exposure | | |
| | | | and remains high at 7 | | |
| | | | days post UV exposure | | |
| Fibrinogen | Fga, Fgb, Fgg | FGG significant increase at | FGG slight decline and | + | i) Regulates clotting |
| | | 48 hours post UV | continues through 7 days. | | ii) Tissue healing |
| | | exposure and return to | FGA, FGB unchanged | | |
| | | baseline at 7 days | | | |
| | | FGA increase at 4 [symbol] hours | | | |
| | | post UV and return to | | | |
| | | baseline at 7 days. | | | |
| | | FGB significant increase at | | | |
| | | 48 hours post UV | | | |
| | | exposure and return to | | | |
| | | baseline at 7 days | | | |
| Apolipoprotein A | Apoa1, Apoa4 | Significant decrease by 48 | Unchanged | − | |
| | | hours post UV exposure | | | |
| | | but resume to baseline at 7 | | | |
| | | days | | | |
| Inter-alpha-trypsin | Itih4 | Significant increase at 48 | Unchanged | + | i) Protease inhibitor |
| inhibitor | | hours post UV exposure | | | |
| heavy Chain B4 | | and returns to baseline at | | | |
| | | 7 days | | | |
| Inter-alpha-trypsin | Itih1 | Significant but slight | Significant slight decrease | | |
| inhibitor | | decrease at 48 hours post | at 48 hours post UV | | |
| heavy Chain H1 | | UV exposure and returns | exposure and stays low at | | |
| | | to baseline at 7 days | 7 days | | |
| Albumin | Alb | Significant decline at 48 | Unchanged | − | 1) Binds fatty acids and |
| | | hours post UV exposure | | | hormones |
| | | and return to baseline at 7 | | | ii) Regulates osmotic |
| | | days | | | pressure in vasculature |
| Transthyretin | Tb | Significant decline at 48 | Unchanged | − | |
| | | hours post UV exposure | | | |
| | | and return to baseline at 7 | | | |
| | | days | | | |
| Thrombospondin-1 | Thbs1 | Significant decrease at 48 | Significant increase at 48 | | |
| | | hours post UV exposure | hours post UV exposure | | |
| | | and returning to baseline | with levels staying high at | | |
| | | at 7 days | 7 days | | |

[symbol]

indicates data missing or illegible when filed

Comparison of protein fold changes between control and 48 hours post UV exposure, as well as control and 1-week post UV exposure, shows a larger number of >4-fold changes in abundance for the mouse plasma proteome. Changes in the naked mole-rat proteome were much more modest (FIG. 14C). A total of 167 proteins were quantified in both species. In many cases the magnitude and direction of protein fold change differed between the two species. Interestingly, 48 hours after exposure to UV irradiation, change in abundance of many proteins varied in the mouse proteome compared to that of the naked mole-rat proteome (reverse/in-sync trends, or no change). For example, changes of 2.8× (fold) for Fibrinogen gamma chain (Fgg), 3.9× for Haptoglobin (Hp), 2.1× for Inter-alpha-trypsin inhibitor heavy chain H4 (Itih4), and −1.2× for Apolipoprotein A-IV (Apoa4), compared to those in naked mole-rat proteome: 1.2× Fgg, 5.3×Hp, no change for Itih4, and −1.5× for Apoa4 (FIG. 14D). Even though samples were collected at the same time of day, it is important to note that proteins seen changing in response to UV exposure, particularly the aforementioned Fgg, Hp, Itih4, and Apoa4, did not have a detectable circadian rhythm (FIG. 13D).

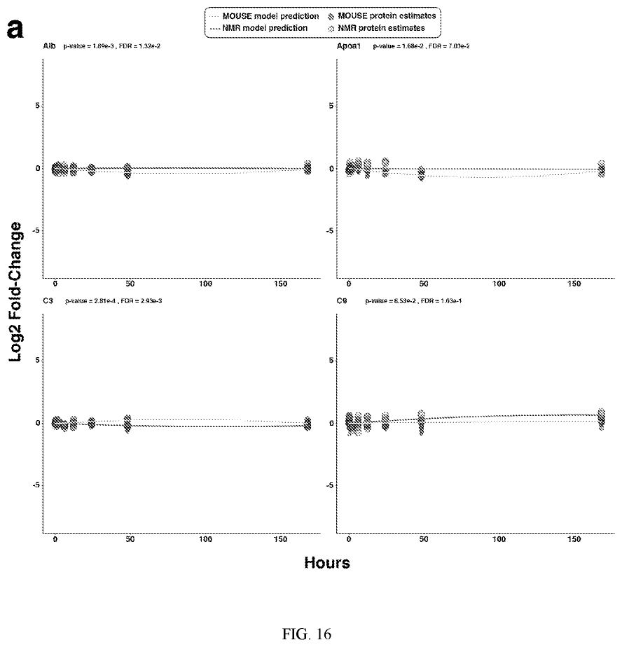

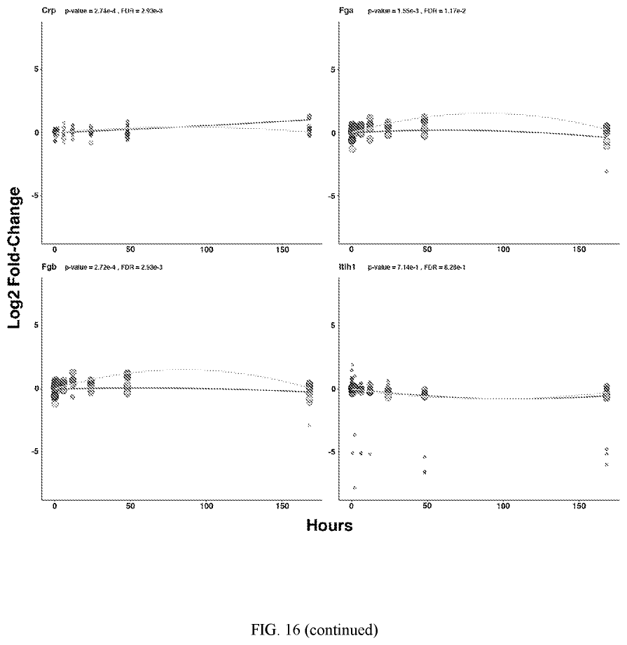

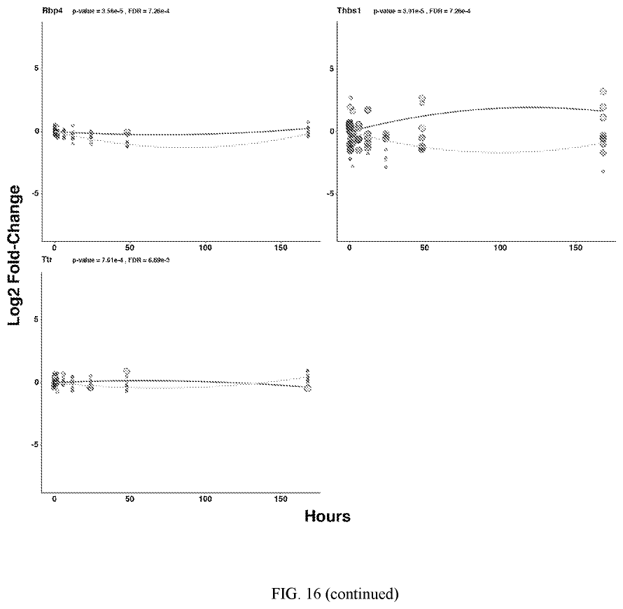

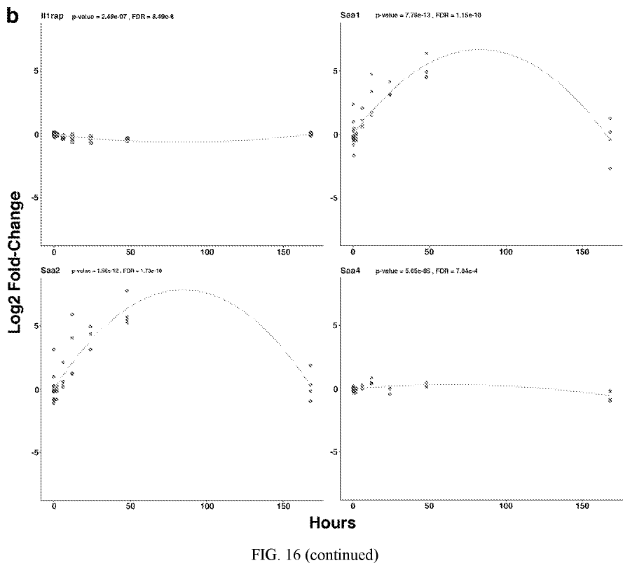

UV radiation induced signs of local low-grade inflammation, are clearly evident in the mouse plasma levels of inflammation-associated proteins, apolipoproteins, and metabolic proteins commonly associated with lipid metabolism and protein stability. High-density lipoprotein (HDL) proteins showed a tendency to change. These included a decline in Apoa1 and Apoa4 suggesting systemic inflammation and dysregulated lipid homeostasis. In addition to these observed changes in the mouse, most of the UV exposure induced changes in plasma proteins over the week-long monitoring period were in keeping with an Acute Phase Response (APR)—a pronounced systemic reaction elicited in response to local trauma, infection, and inflammation. These APR responsive proteins, commonly known as Acute Phase Proteins (APPs), showed the most pronounced changes at the 48 hrh time point. Both negative increases and decreases in APP protein levels were evident. A marked decline in abundance of albumin (Alb), as well as a transthyretin (Ttr), Apoa1, Interleukin-1 receptor accessory protein (Il1rap), and retinol binding protein (Rbp4), were evident with the abundance in these negative APPs declining by 20-53% (FIG. 16). In contrast, an increase in abundance was observed in mouse levels of C-reactive protein (Crp), Hp, Fgg, Itih4, and complement factor 3, (FIG. 14D, FIG. 16) with >30-fold increases observed in both Serum amyloid A1 (Saa1) and Saa2 at the 48 hr time point (FIG. 16).

Although it was speculated that mole-rats would be more sensitive to UV, having lived in an environment devoid of UV light for more than 18 million years, surprisingly acute UV exposure has little effect on their plasma proteins. None of the negative APPs significantly changed over the time course post UV exposure (Table 7). Similarly, abundance of Apoa1, Itih4, and fibrinogen proteins were unaffected by UV. Hp, complement factors (C3 and C9) and Crp abundance changed after UV exposure, but their pattern of response was markedly different to that of mice (Table 7). Generally, reactions to UV were modest, and unlike mice, changes in the abundance levels of these proteins seven days after exposure were similar to that at 48 hr after exposure. For example, compared to the mouse response, Crp showed a significant but small change 48 hr after UV treatment and continued to show a modest increase at the one-week time point (FIG. 16).

### Example 6: Optimization of a Multiplexed Proteome Profiling Platform (MP3) Using a Single Population of Microbead

Two experimental methods were evaluated as alternatives to standard mass spectrometer data acquisition: (1) Native-MP3 binding+AutoMP3 with TMT labeling, and (2) Native-MP3 binding+label-free AutoMP3.

### I. Native-MP3 Binding & Proceeding AutoMP3 Stages

### MP3-Native

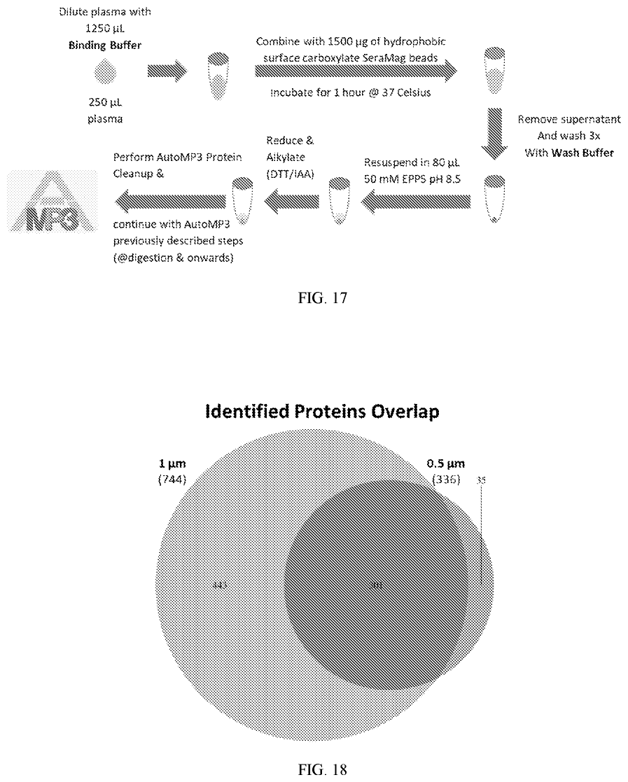

As depicted in FIG. 17 an exemplary sample preparation workflow for Native-MP3 involves multiple steps. A binding and wash buffer (modified TE Buffer) was made by combining the 10 mM Tris, 1 mM disodium EDTA, pH 8.0 buffer (Sigma Aldrich, #93283-100ML) with the appropriate volume of 5 M KCl solution and 2% CHAPS solution to reach final solution of approximately 10 mM Tris, 1 mM disodium EDTA, 150 mM KCl, 0.05% CHAPS, pH 8.0. Prior to handling samples, 1500 μg of 1 μm average diameter hydrophobic surface carboxylated SpeedBead SeraMag beads (Cytiva, #44152105050350) were aliquoted into Axygen Maxymum Recovery 2 mL tubes (#MCT-200-L-C) per sample, and washed 3× with water via magnet immobilization with supernatant removed. Each 250 μL plasma sample was then diluted 6× with the addition of 1250 μL modified TE buffer, and the resultant mixture was added to the tube containing 1500 μg magnetic particles. The modified TE binding buffer emulated native conditions for plasma proteins. The entire mixture was then gently but thoroughly pipette mixed. The mixture comprising the plasma sample and magnetic microparticles was incubated for 1 hour at 300 rpm, 37° C. The incubation of the mixture induced preferential binding to bead surface of native proteins and complexes of native proteins. The preferential binding of proteins (referred to as “enriched proteins”) decreases the abundance of various highly abundant proteins in plasma, allowing a greater number of proteins to be identified compared to a regular shotgun proteomics experiment. Following incubation, the magnetic particles were immobilized on a magnet for 3 minutes and the supernatant (containing unbound plasma proteins) was removed. The enriched proteins on beads were subsequently washed using the modified TE Buffer by adding 1 mL of modified TE Buffer, mixing the magnetic particles gently through pipette mixing and incubating at 300 rpm for 3 minutes at 37° C. Supernatant was then removed and 3 washes were performed. The protein-bound beads were gently resuspended in 80 μL of 50 mM EPPS pH 8.5. The enriched proteins on beads were further processed for proteome profiling using either TMT (with AutoMP3) or with label-free proteomics.

### AutoMP3 Post Native-MP3

Proteins obtained from the Native-MP3 workflow were reduced by the addition of DTT (5 mM final concentration, 300 rpm, 30 mins, RT). This was followed by alkylation using iodoacetamide (15 mM final concentration, 300 rpm, RT, 30 mins in the dark). The alkylation reaction was subsequently quenched by the addition of DTT (final concentration 5 mM, RT). Protein cleanup was performed by the addition of 100% acetonitrile to reach 75% concentration to re-bind the protein sample back to the beads, with an 18-minute incubation period (RT). Particles were then immobilized on the magnet for 2 minutes, supernatant removed, followed by two 100 μL 70% ethanol washes, with a final 100 μL 100% acetonitrile wash. The sample was then resuspended in 90 μL 100 mM EPPS 10 mM CaCl2 pH 8.5 buffer, followed by protein digestion by adding 3.33 μg of LysC/Trypsin protease mixture in 10 μL 100 mM EPPS, 10 mM CaCl2 pH 8.5 buffer. Digestion was performed overnight at 37 degrees Celsius, 600 rpm. Evaluation of recovered peptide material was then performed using Pierce's BCA Assay kit as per manufacturer's instructions.

### II. AutoMP3 Label-Free Stage

Post digestion, samples were immobilized for 2 minutes and the peptides (supernatant) moved to a fresh batch of 1:1 mixed hydrophobic: hydrophilic, 1000 μg SeraMag SpeedBeads (Cytiva, #44152105050350 & #45152105050350) at no more than 100 μL aqueous volume to bind and cleanup with magnetic beads in 2 mL tubes or 96-well 2 mL plate. Binding was achieved through the addition of 1900 μL 100% acetonitrile (>=95% final ACN concentration), and incubated for 18 minutes, no shaking. Bead-bound samples were washed 1× with 1000 μL 100% ACN. The portions were eluted 3× with 20 μL 5% ACN, 2 minutes 600 rpm, followed by magnet immobilization, elutions re-combined and dried down in a speedvac overnight.

### III. AutoMP3 TMT Multiplex Stage

Post digestion, following removal from the magnet immobilized beads, TMT labeling was performed by aliquoting out 5 μg peptides per sample (based on peptide BCA measurements) into 80 μL digestion buffer. TMT reagent was used at a ratio of 8:1. Excess TMT was quenched with 5% hydroxylamine to a final concentration of 0.3% v/v. Samples per plex were then combined, pipette mixed, and split back out into portions containing no more than 100 μL aqueous volume to bind and cleanup with magnetic beads in 2 mL tubes or 96-well 2 mL plate. Binding was achieved through addition of 1900 μL 100% acetonitrile (>=95% final ACN concentration), and incubated for 18 minutes, no shaking. Bead-bound peptides were washed 1× with 1000 μL 100% ACN. The portions were eluted 3× with 20 μL 5% ACN, 2 minutes 600 rpm, followed by magnet immobilization. Elutions and plex ‘portions’ were re-combined and dried down in a speedvac overnight.

### IV. Mass Spectrometer Data Acquisition

Samples were analyzed either on an Orbitrap Fusion Lumos mass spectrometer (Thermo Fisher Scientific) coupled to an Easy-nLC1200 (Thermo Fisher Scientific) or on an Orbitrap Eclipse mass spectrometer with Field Asymmetric Ion Mobility Spectrometry unit (FAIMSpro) with Real Time Search (RTS), (Thermo Fisher Scientific) coupled to an UltiMate 3000 (Thermo Fisher Scientific) as indicated. For both label-free and TMT multiplexed peptides, online separation was performed on an IonOpticks Aurora microcapillary column (75 μm inner diameter, 25 cm long, C18 resin, 1.6 μm, 120 Å). The total LC-MS run length for each sample was 180 min including a 165 min gradient from 8 to 30% ACN in 0.1% formic acid for TMT 16-plex multiplexed samples. For label-free experiments the total run length was 85 minutes, with 75-minute gradient from 1 to 25% ACN in 0.1% formic acid. The flow rate was 300 nL/min, and the column was heated at 60° C. Data was collected using the data-dependent acquisition (DDA) mode.

### Label-Free Data Acquisition Mass Spectrometry Method on an Orbitrap Fusion Lumos

A high resolution MS1 scan in the Orbitrap (m/z range 375-1,500, 120k resolution, Automatic Gain Control (AGC) 1×10\hat{ }6, max injection time 100 ms, RF for ion funnel 30%) was collected, from which the top 10 precursors were selected for MS2 followed by synchronous precursor selection (SPS) MS3 analysis. For MS2 spectra, ions were isolated with the quadrupole mass filter using a 0.5 m/z isolation window. The MS2 scan was performed in the quadrupole ion trap (Collision Induced Dissociation (CID), AGC 2×10\hat{ }4, normalized collision energy 30%, max injection time 35 ms) or with Higher-energy collisional dissociation (HCD) in the ion trap, AGC 2×10\hat{ }4, normalized collision energy 20%, max injection time 35 ms.

### TMT Data Acquisition Mass Spectrometry Method on Orbitrap Eclipse

RTS and FAIMS Pro were combined with DDA. RTS utilized a uniprot mouse database. Four FAIMS Pro compensation voltages were used: −40, −50, −60, and −70 Volts. Each of the four experiments had a 1.25 seconds cycle time. A high resolution MS1 scan in the Orbitrap (m/z range 400-1,600, 120k resolution, “Standard” AGC, “Auto” maximum injection time, ion funnel RF of 30%) was collected, from which the top 10 precursors were selected for MS2 followed by SPS MS3 analysis. For MS2 spectra, ions were isolated with the quadrupole mass filter using a 0.7 m/z isolation window. The MS2 product ion population was analyzed in the quadrupole ion trap (CID, “Standard” AGC, normalized collision energy 35%, “Auto” max injection time) and the MS3 scan was analyzed in the Orbitrap (HCD, 50k resolution, 200% “Normalized AGC Target”, max injection time 200 ms, normalized collision energy 45%). Up to ten fragment ions from each MS2 spectrum were selected for MS3 analysis using SPS. The mass tolerance for the target masses were 10 ppm (low and high).

V. Comparison of Data Acquired from Native MP3 and Regular MP3

Method Comparison and Optimization Samples. For all methods mouse plasma from naive C57/B6 mice are used. Mouse plasma was collected in-house from C57/B6 mice.

### Example 7: Comparisons of Native-MP3 and Regular-MP3 Methodologies

Samples are prepared and analyzed as described above in Examples 1-6.

### I. Comparison of Different Sized Beads Using Label-Free Native-MP3

Using a Native-MP3 coupled with transition to label-free protocol (see Example 6), 250 μL plasma was processed using either 0.5 μm or 1 μm carboxylated, solid core magnetic particles for the 1 hour incubation. One μg of recovered peptide material was injected over a 85 minute gradient on EASY-nLC1200 with 25 cm IonOpticks Aurora column on a Lumos Fusion (no FAIMS: no RTS). Data was searched with Comet using a conservative SwissProt FASTA database with reverse hits and common contaminants, and filtered to 1% FDR at the protein level.

As shown in Table 8, using the Native-MP3 workflow, a greater number of proteins was identified with 1 μm carboxylated beads than 0.5 μm carboxylated beads.

TABLE 8
Total proteins, total peptides, and unique peptides identified
for Native-MP3 performed on different sized beads.

| | Total | Unique | Total |
|---|---|---|---|
| | Peptides | Peptides | Protein |
| 1 μm, carboxyl, solid core | 13331 | 4687 | 744 |
| 0.5 μm, carboxyl, solid core | 14682 | 2548 | 336 |

Additionally, there was a large overlap of identified proteins was detected between Native-MP3 performed on different sized beads (FIG. 18).

### II. Comparison of Label-Free Regular-MP3 Versus Single-Bead Based Native-MP3

Using Native-MP3 coupled with transition to label-free protocol (see Example 6), 250 μL plasma was processed using 1 μm carboxylated, solid core magnetic particles for the 1 hour incubation, and the natively bound portion was then prepared as previously described. Unbound material (supernatant) was removed. Additionally, 100 μg samples were prepared using label-free Regular-MP3 (see Examples 1-5). For both samples (label-free MP3 and label-free Regular-MP3), 1 μg of recovered label-free peptide material was injected over a 85 minute gradient on an EASY-nLC1200 with 25 cm IonOpticks Aurora column on a Lumos Fusion (no FAIMS: no RTS). Data was searched with Comet using a conservative SwissProt only FASTA database with reverse hits and common contaminants, and filtered to 1% FDR at the protein level.

As shown in Table 9, using the single-bead Native-MP3 workflow, a greater number of proteins was identified than with the label-free Regular-MP3 workflow.

TABLE 9
Total proteins, total peptides, and unique peptides identified
for single-bead Native-MP3 using 1 μm carboxylated, solid
core magnetic particles and label-free Regular-MP3.

| | Total | Unique | Total |
|---|---|---|---|
| | Peptides | Peptides | Protein |
| REGULAR-MP3 | 8547 | 2540 | 340 |
| NATIVE-MP3 | 13331 | 4687 | 744 |

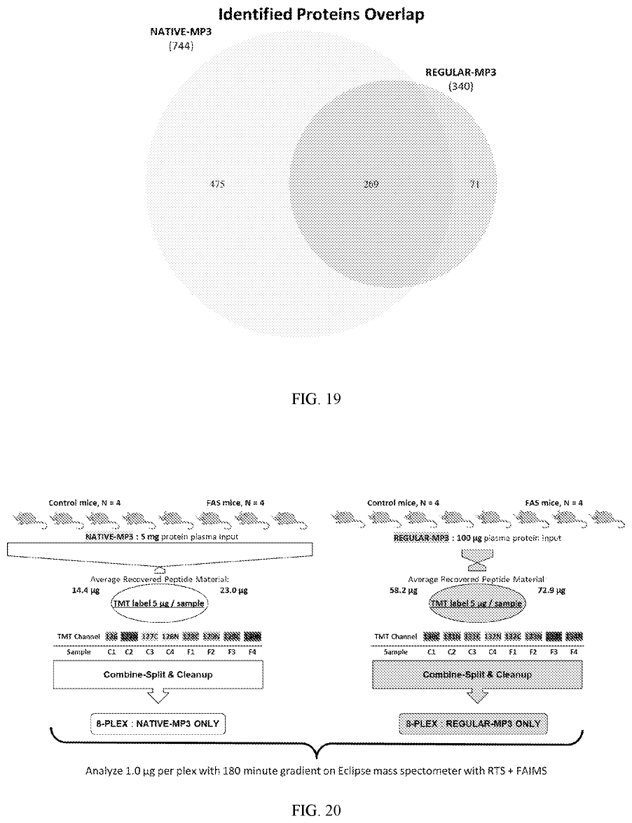

Additionally, there was a large overlap of identified proteins was detected between Native-MP3 and Regular-MP3, with approximately a 2-fold larger identification rate for MP3-Native (FIG. 19).

### Example 8: Comparisons of Native-MP3 and Regular MP3 Methodologies

An experimental design was created to compare workflow methodologies for Native-MP3 and Regular-MP3 (FIG. 20). Plasma from 8 mice (4 WT and 4 LPR/FAS mutant mice) was processed using Native-MP3 and Regular-MP3 in two separate TMT plexes. This allowed for a comparison of both the proteins identified by the two different methods as well as allowing a comparison of the differentially expressed changes between the control and mutant mice.

Using Native-MP3 coupled with transition to TMT 16-plex multiplexed AutoMP3 protocol, 250 μL plasma, in 4 replicates per Control or FAS mutant mouse plasma were processed using 1 μm carboxylated, solid core magnetic particles. Additionally, 2 μL of plasma (or approximately 120-100 μg) was processed with the same experimental setup using Regular-MP3 workflow. For both methods, 5 μg of peptide material per sample were TMT labeled.

For mass spectrometer analysis, an 8-plex was created by pooling 1 μg TMT labeled peptides for the samples from the Native-MP3 method, and similarly a separate 8-plex with the Regular-MP3 method. For each plex, 1 μg injection was injected over a 180 minute gradient on UltiMate 3000 LC with 25 cm IonOpticks Aurora column on a Orbitrap Eclipse (with FAIMS & RTS). Data was searched with Comet using a SwissProt mouse protein FASTA database with reverse hits and common contaminants, and filtered to 1% FDR at the protein level.

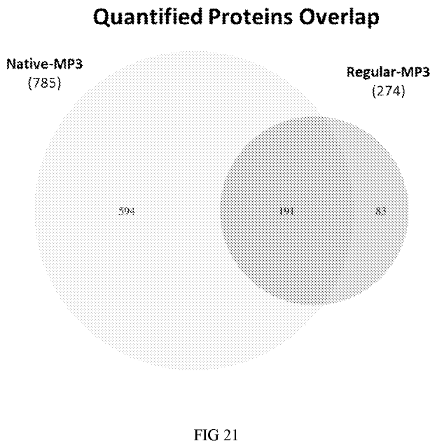

As shown in Table 10, the number of identified proteins using the Native-MP3 method was approximately 3× higher compared to the Regular-MP3 method. There was a large overlap of quantified proteins that were detected between Native-MP3 and Regular-MP3 (FIG. 21).

TABLE 10
Total proteins, total peptides, and unique peptides identified
for single-bead based Native-MP3 using 1 μm carboxylated,
solid core magnetic particles and single-bead Regular-MP3
using 1 μm carboxylated, solid core magnetic particles.

| | Total | Unique | Total |
|---|---|---|---|
| | Peptides | Peptides | Protein |
| NATIVE-MP3 ONLY | 14012 | 6073 | 931 |
| REGULAR-MPS ONLY | 73730 | 3186 | 311 |

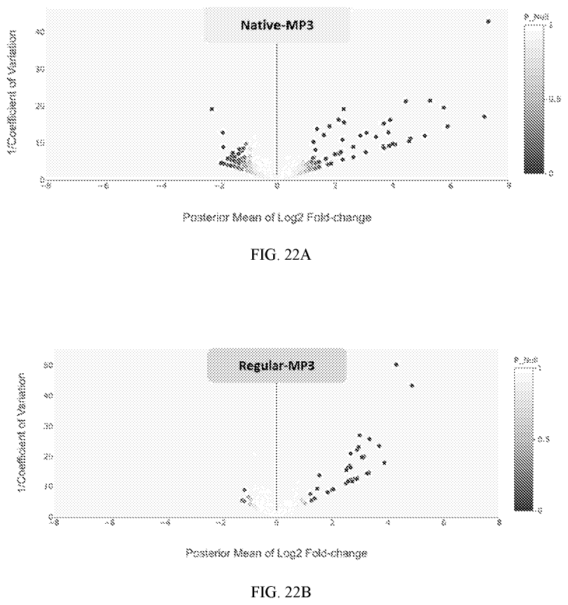

A greater number of differentially expressed protein changes were detected for Native-MP3 (FIG. 22A) between WT and mutant (FAS) mice compared to regular-MP3 (FIG. 22B). differentially expressed proteins between WT and mutant (FAS) mouse.

As shown Table 11, the number of differentially expressed proteins using Native-MP3 is approximately 2.5-fold higher compared to Regular-MP3. For differential expression changes greater than 40%, there are approximately 3-fold more proteins identified using Native-MP3 compared to Regular-MP3. At greater than 4-fold changes the number of proteins quantified are approximately the same using Native-MP3 compared to Regular-MP3. Native-MP3 allows us to quantify more differentially expressed proteins than Regular-MP3, especially those with smaller fold changes.

TABLE 11
Differentially expressed proteins for
Native-MP3 and Regular-MP3 workflows.

| | Native-MP3 | Regular-MP3 |
|---|---|---|
| Quantified Protein IDs | 618 | 246 |
| >40% | 272 | 81 |
| >4 fold | 32 | 28 |

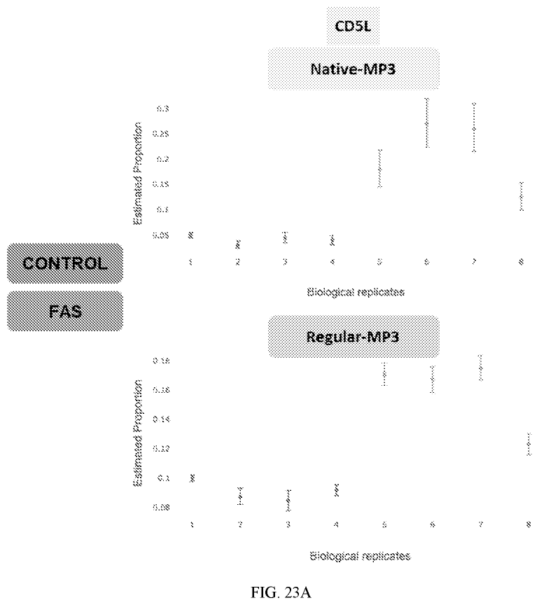

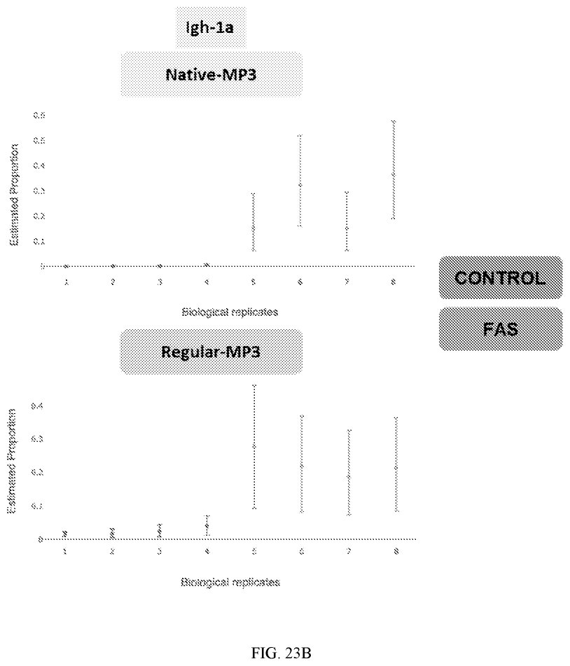

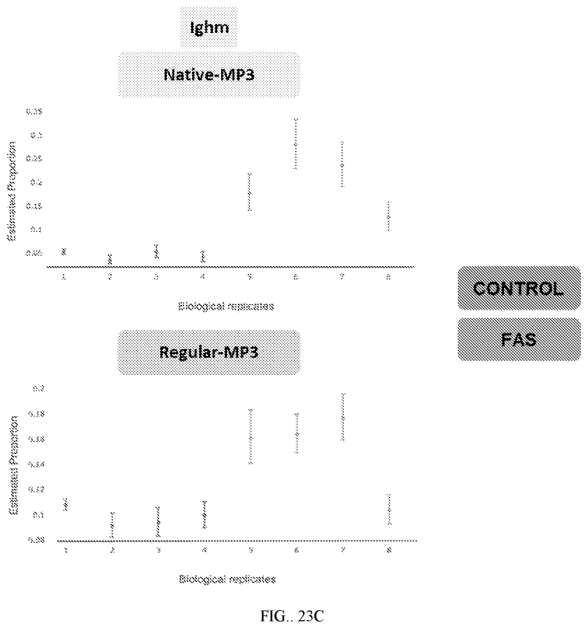

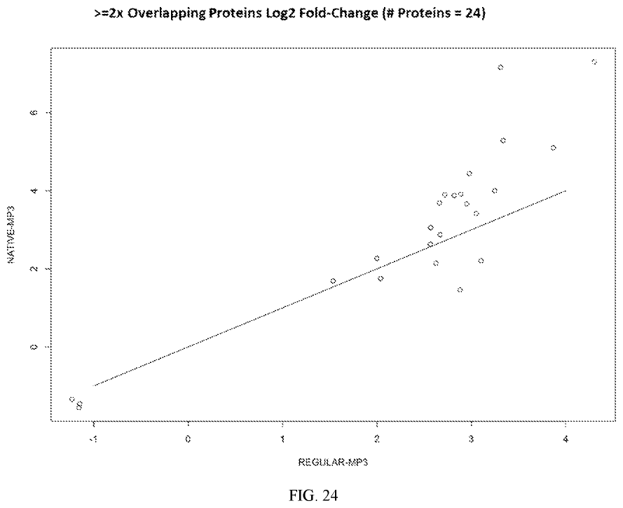

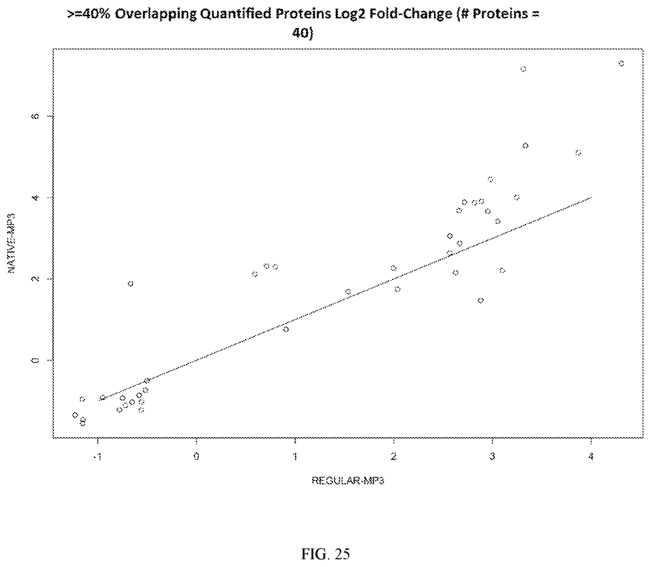

Similar patterns and fold changes were observed for differentially expressed proteins detected in both Native-MP3 and Regular-MP3. Examples of proteins which exhibited similar patterns and fold changes include CD5L (FIG. 23A), Igh-1a (FIG. 23B), and Ighm (FIG. 23C). Comparing quantified protein expression of proteins having greater than 2-fold protein fold changes for WT versus mutant (FAS) mice, the Native-MP3 method trended to have larger magnitude changes for some proteins, but similar expression for other proteins compared to the Regular-MP3 method (FIG. 24). Additionally, comparing quantified protein expression of proteins having greater than 40% changes for WT versus mutant (FAS) mice, the Native-MP3 method trended to have larger magnitude changes for some proteins, but similar expression for other proteins compared to the Regular-MP3 method (FIG. 25).

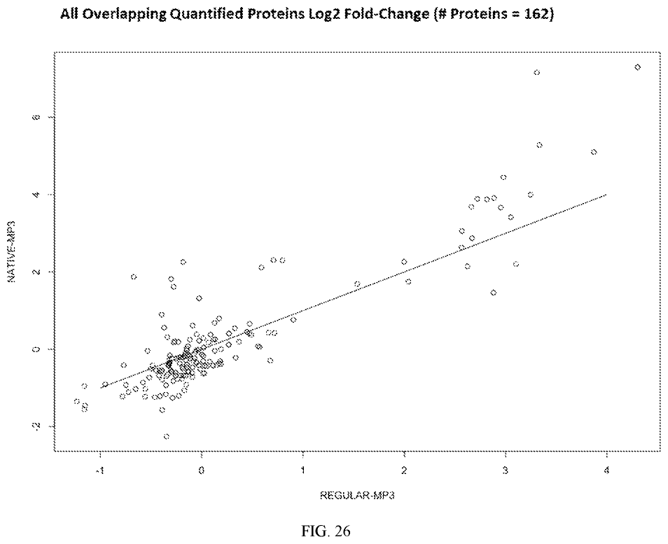

Comparing quantified protein expression of all overlapping quantified proteins for WT versus mutant (FAS) mice, the majority of overlapping quantified proteins trended to have similar magnitude, between Native-MP3 and Regular-MP3, especially when the change was small (FIG. 26). In addition, for the larger magnitude changes, the detected change trended to be larger for the Native-MP3 method compared to the Regular-MP3 method (FIG. 26).

### INCORPORATION BY REFERENCE

The entire disclosure of each of the patent documents and scientific articles cited herein is incorporated by reference for all purposes.

### EQUIVALENTS

The disclosure can be embodied in other specific forms with departing from the essential characteristics thereof. The foregoing embodiments therefore are to be considered illustrative rather than limiting on the disclosure described herein. The scope of the disclosure is indicated by the appended claims rather than by the foregoing description, and all changes that come within the meaning and range of equivalency of the claims are intended to be embraced therein.

1. A method for quantifying or identifying two or more peptides or polypeptides, comprising the steps of:
(a) Combining two or more peptides or polypeptides into one plex;
(b) Splitting the plex of peptides or polypeptides into two or more portions;
(c) Re-combining the portions into one plex; and
(d) Analyzing the peptides or polypeptides so as to obtain mass spectrometry data;
thereby quantifying and identifying peptides or polypeptides.

2. A method for quantifying or identifying two or more chemically isotopic labeled peptides or polypeptides, comprising the steps of:
(a) Combining two or more labeled peptides or polypeptides into one plex;
(b) Splitting the plex of labeled peptides or polypeptides into two or more portions;
(c) Re-combining the portions into one plex; and
(d) Analyzing the labeled peptides or polypeptides so as to obtain mass spectrometry data;
thereby quantifying and identifying two or more chemically isotopic labeled peptides or polypeptides.

3. The method of claim 1, wherein the method further comprises mixing the two or more peptides or polypeptides in the single plex prior to the splitting in step (b).

4. The method of claim 1, wherein the method further comprises:
(i) normalizing the concentration values of the two or more peptides or polypeptides to a reference sample prior to step (a);
(ii) clean-up of each portion prior to step (c), wherein the clean-up method is selected from the group consisting of peptide precipitation, in-solution (IS, in-StageTip (IST), Single-Pot Solid-phase enhanced Sample Preparation (SP3), filter-aided sample preparation (FASP), S-Trap, or SepPak;
(iii) generating a bridge;
(iv) analyzing of step (d) comprises protein identification using high-field asymmetric waveform ion mobility (FAIMS Pro) and Real Time Search (RTS); and/or
(v) performing the method on an automated liquid handling robot.

5-7. (canceled)

8. The method of claim 2, wherein the chemically isotopic labeled peptides or polypeptides are isobarically labeled or otherwise isotope incorporated.

9. (canceled)

10. The method of claim 2, wherein the method further comprises mixing the two or more peptides or polypeptides in the single plex prior to the splitting in step (b).

11. (canceled)

12. The method of claim 10, wherein the method further comprises:
(i) normalizing the concentration values of the two or more peptides or polypeptides to a reference sample;
(ii) clean-up of each portion prior to step (cd), wherein the clean-up method is selected from the group consisting of peptide precipitation, in-solution (IS, in-StageTip (IST), Single-Pot Solid-phase enhanced Sample Preparation (SP3), filter-aided sample preparation (FASP), S-Trap, or SepPak;
(iii) the analyzing of step (d) comprises protein identification using high-field asymmetric waveform ion mobility (FAIMS Pro) and Real Time Search (RTS)
(iv) generating a bridge; and/or
(v) performing the method on an automated liquid handling robot.

13-18. (canceled)

19. A method for obtaining a plurality of enriched peptides or polypeptides in a sample, comprising the steps of:
(a) Contacting the sample with beads comprised of a single type of bead;
(b) Separating a portion of the sample comprising protein-bound beads from a portion of the sample comprising unbound proteins; and
(c) Resuspending the portion of the sample comprising protein-bound beads, thereby obtaining a plurality of enriched peptides or polypeptides in the sample.

20. The method of claim 19, wherein the sample is selected from the group consisting of whole blood, plasma, serum, urine, saliva, tears, spinal fluid, synovial fluid, cell lysate, tissue lysate, exosomes and individual cell organelles.

21. (canceled)

22. The method of claim 19, wherein the beads are
(i) magnetic;
(ii) about 0.1 μm to 10 μm size in nanometers or 1 nm to 1000 μm size micrometers in diameter; and/or
(iii) incubated with the sample at physiological conditions.

23-26. (canceled)

27. The method of claim 19, wherein the method is performed at physiological conditions, a pH of about 7.4, and/or a temperature of about 37 degrees Celsius.

28. The method of claim 19, wherein the plurality of enriched peptides or polypeptides comprises one or more protein complexes and/or the method further comprises reducing the plurality of enriched peptides or polypeptides.

29. The method of claim 19, wherein the method separating of step (b) comprises magnetic immobilization to separate a portion of protein-bound beads from a portion of unbound plasma proteins and unbound beads in the sample.

30. (canceled)

31. The method of claim 19, wherein the method further comprises alkylating the plurality of enriched peptides or polypeptides.

32. The method of claim 19, wherein the method further comprises:
(i) normalizing the concentration values of the two or more peptides or polypeptides to a reference sample;
(ii) clean-up of the plurality of enriched peptides or polypeptides;
(iii) digesting the plurality of enriched peptides or polypeptides, optionally wherein resuspending the portion of the sample comprising protein-bound beads includes the digesting or the digesting comprises adding protease to the peptides or polypeptides;
(iv) generating a bridge; and/or
(v) performing the method on an automated liquid handling robot.

33-35. (canceled)

36. The method of claim 19, wherein the method further comprises isobarically labeling the plurality of enriched peptides or polypeptides.

37-38. (canceled)

39. A method for quantifying or identifying peptides or polypeptides, comprising the steps of:
(a) Obtaining a plurality of labeled peptides or polypeptides according to the method of claim 36;
(b) Aliquoting the labeled peptides or polypeptides into two or more portions;
(c) Combining the two or more labeled portions into one plex;
(d) Splitting the plex of labeled peptides or polypeptides into two or more portions for clean-up;
(e) Clean-up of each portion;
(f) Re-combining the portions into one plex; and
(g) Analyzing the peptides or polypeptides so as to obtain mass spectrometry data, thereby quantifying or identifying peptides or polypeptide.

40-43. (canceled)

44. A method for quantifying or identifying peptides or polypeptides in a sample, comprising the steps of:
(a) Obtaining the plurality of enriched peptides or polypeptides of claim 19;
(b) Incorporating isotopic labels onto each peptide or polypeptide in the portion of the sample comprising protein-bound beads;
(c) Aliquoting the labeled peptides or polypeptides into two or more portions;
(d) Combining the two or more isotopically labeled peptides or polypeptides into one plex;
(e) Splitting the plex of labeled peptides or polypeptides into two or more portions for clean-up;
(f) Re-combining the portions into one plex; and
(g) Analyzing the peptides or polypeptides so as to obtain mass spectrometry data, thereby quantifying or identifying peptides or polypeptides in a sample.

45. The method of claim 44, wherein the method further comprises mixing the two or more peptides or polypeptides prior to the splitting in step (e).

46-53. (canceled)

54. A method for quantifying or identifying peptides or polypeptides in a sample, comprising the steps of:
(a) Obtaining a plurality of enriched peptides or polypeptides according to the method of claim 19; and
(b) Analyzing the peptides and polypeptides so as to obtain mass spectrometry data;
thereby quantifying or identifying peptides or polypeptides in a sample.

55-62. (canceled)
